(19) United States
(12) Patent Application Publication
(10) Pub. No.: US 2024/0336650 A1
(43) Pub. Date: Oct. 10, 2024

(54) PLASMA FRACTIONATION BY CONTINUOUS EXTRACTION

(71) Applicant: CSL Behring AG, Bern (CH)

(72) Inventor: Ibrahim EL MENYAWI, Bern (CH)

(73) Assignee: CSL Behring AG, Bern (CH)

(21) Appl. No.: 18/563,232

(22) PCT Filed: May 26, 2022
(86) PCT No.: PCT/EP2022/064372

§ 371 Date: Nov. 21, 2023

(30) Foreign Application Priority Data
May 26, 2021 (AU) 2021901577

(51) Int. Cl. C07K 1/36; B01D 21/01; B01D 61/14; B01D 63/16; B01D 65/02; B01D 69/06; B01D 71/02

(52) U.S. Cl. CPC C07K 1/36; B01D 21/01; B01D 61/145; B01D 63/16; B01D 65/02; B01D 69/06; B01D 71/02; B01D 2313/501; B01D 2315/02; B01D 2315/10; B01D 2315/17

(57) ABSTRACT

The invention relates to a method for the purification of immunoglobulin and/or albumin from a sample of plasma, the method comprising mixing a sample of plasma with a medium chain fatty acid, and recovering the soluble immunoglobulin from the mixture.

### CROSS REFERENCE TO EARLIER APPLICATION

This application is the U.S. National Stage of International Patent Application No. PCT/EP2022/064372, filed May 26, 2022, which claims priority to Australian Provisional Application No. 2021901577, filed May 26, 2021, the entire contents of which are hereby incorporated by reference in their entirety.

### FIELD OF THE INVENTION

The present invention relates to a method and system for extracting immunoglobulins (Ig) such as immunoglobulin G (IgG), from plasma.

### BACKGROUND OF THE INVENTION

The demand for purified proteins such as specific antibodies has increased considerably. Such purified proteins can be used for therapeutic and/or diagnostic purposes.

Human blood plasma has been industrially utilized for decades for the production of widely established and accepted plasma-protein products such as human albumin (HSA), immunoglobulin (IgG), clotting factor concentrates (clotting Factor VIII, clotting Factor IX, prothrombin complex etc.) and inhibitors (antithrombin, C1-inhibitor etc.). In the course of the development of such plasma-derived drugs, plasma fractionation methods have been established, leading to intermediate products enriched in certain protein fractions, which then serve as the starting composition for plasma-protein product/s. Typical processes are reviewed in e.g. Molecular Biology of Human Proteins (Schultze H. E., Heremans J. F.; Volume I: Nature and Metabolism of Extracellular Proteins 1966, Elsevier Publishing Company; p. 236-317). These kinds of separation technologies allow for the production of several therapeutic plasma-protein products from the same plasma donor pool. This is economically advantageous over producing only one plasma-protein product from one donor pool, and has therefore been adopted as the industrial standard in blood plasma fractionation.

One example of this type of fractionation process, cold ethanol fractionation of plasma, was pioneered by E. J Cohn and his team during World War II, primarily for the purification of albumin (Cohn E J, et al. 1946, J. Am. Chem. Soc. 62:459-475). The Cohn fractionation process involves increasing the ethanol concentration in stages, from 0% to 40%, while lowering the pH from neutral (pH 7) to about 4.8, resulting in the precipitation of albumin. Whilst Cohn fractionation has evolved over the past 70 years or so, most commercial plasma fractionation processes are based on the original process or a variation thereof (e.g. Kistler/Nitschmann), exploiting differences in pH, ionic strength, solvent polarity and alcohol concentration to separate plasma into a series of major precipitated protein fractions (such as Fractions I to V in Cohn).

Variations to the Cohn Fractionation process have been developed with the aim of improving polyvalent IgG recovery. For example Oncley and co-workers used Cohn Fractions II+III as a starting material with different combinations of cold ethanol, pH, temperature and protein concentration to those described by Cohn, to produce an active immune globulin serum fraction (Oncley et al., (1949) J. Am. Chem. Soc. 71, 541-550). Today, the Oncley method is the classic method used for production of polyvalent IgG. Nevertheless, it is known that approximately 5% of gamma-globulins (antibody-rich portion) is co-precipitated with Fraction I and about 15% of the total gamma-globulin present in plasma is lost by the Fraction II+III step (See Table III, Cohn E J, et al. 1946, J. Am. Chem. Soc. 62:459-475). The Kistler/Nitschmann method aimed to improve IgG recovery by reducing the ethanol content of some of the precipitation steps (Precipitation B vs Fraction III). The increased yield, however, is at the expense of the purity (Kistler & Nitschmann, (1962) Vox Sang. 7, 414-424).

Initially, immunoglobulin G (IgG) preparations derived from these fractionation processes were successfully used for the prophylaxis and treatment of various infectious diseases. However as ethanol fractionation is a relatively crude process the IgG products contained impurities and aggregates to an extent that they could only be administered intramuscularly. Since that time additional improvements in the purification processes have led to IgG preparations suitable for intravenous (called IVIg) and subcutaneous (called SCIg) administration.

It has been estimated that approximately 30 million liters of plasma were processed worldwide in 2010, providing a range of therapeutic products including about 500 tonnes of albumin and 100 tonnes of IVIg. The IVIg market accounts for about 40-50% of the entire plasma fractionation market (P. Robert, Worldwide supply and demand of plasma and plasma derived medicines (2011) J. Blood and Cancer, 3, 111-120). Thus, with demands for IVIg remaining strong (along with increasing demands for SCIg) there remains a need to improve immunoglobulin recoveries from plasma and related fractions. Preferably, this must be achieved in a way that ensures the recovery of other plasma derived therapeutic proteins is not adversely affected.

From a commercial perspective, the initial fractionation processes are critical to the overall production time and costs associated with the production of a therapeutic protein, particularly plasma derived proteins, since the subsequent purification steps will depend on the yield and purity of the protein(s) of interest within these initial fractions. Whilst several variations of the cold ethanol fractionation process have been developed for plasma derived protein in order to improve protein yield at lower operating costs, higher protein yields are typically associated with lower purity.

There is a need for an improved method and system for the industrial scale production of proteins from blood-derived plasma or serum and which meet stringent safety standards. The currently used downstream technologies are relatively expensive and their yield is not optimal. Therefore, there is a crucial need to develop more efficient and economic methods for the extraction and purification of proteins such as immunoglobulins, from plasma.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention is based on the finding that it is possible to obtain a relatively pure preparation of immunoglobulins, particularly IgG, directly from plasma, and without the need for ethanol fractionation steps.

Further, the present invention is based on the finding that it is also possible to obtain a relatively pure preparation of immunoglobulins, particularly IgG, and albumin directly from plasma, and without the need for ethanol fractionation steps.

Further again, the present invention is based on the finding that it is also possible to obtain a relatively pure preparation of albumin directly from plasma, and without the need for ethanol fractionation steps.

Accordingly, in a first aspect, the present invention provides a method for obtaining a solution of immunoglobulins, the method comprising:
contacting a sample of blood-derived plasma with a medium chain fatty acid, in conditions for enabling the selective precipitation of albumin from the sample,
wherein the conditions comprise a pH range of between about 4.6 to about 5.0;
thereby forming a solution of immunoglobulins.

Preferably, the conditions also comprise a conductivity of about 5 mS/cm to about 12 mS/cm, preferably 8 mS/cm to about 12 mS/cm.

In a second aspect, the present invention provides a method for the purification of immunoglobulins, the method comprising:
contacting a sample of blood-derived plasma with a medium chain fatty acid, in conditions comprising a pH range of between about 4.6 to about 5.0, for enabling the selective precipitation of albumin from the sample,
thereby forming a suspension comprising a soluble protein-containing component comprising immunoglobulins and an insoluble protein-containing component comprising albumin;
separating the soluble protein-containing component from the insoluble protein-containing component to obtain a solution of purified immunoglobulins.

The step of separating the soluble protein-containing component from the insoluble protein-containing component preferably comprises subjecting the suspension to continuous extraction filtration. Preferably, the step of separating the soluble protein-containing component from the insoluble protein-containing component comprises:
feeding the suspension into a filtration unit comprising a dynamic filter element adapted to produce a first retentate and a first filtrate;
recovering the first filtrate.

The first retentate comprising the insoluble protein-containing component will typically be enriched for albumin and the first filtrate comprising the soluble protein-containing component will be enriched for immunoglobulins.

The first filtrate enriched in immunoglobulin may be subjected to a concentration step prior to further processing. The concentration step may comprise a continuous concentration process whereby the first filtrate is fed into a second filtration unit comprising a cross flow filter element or TFF, adapted to produce a second retentate enriched in immunoglobulins; and a second filtrate depleted of immunoglobulins. Optionally, the second filtrate depleted of immunoglobulins may be streamed back into the first tank and/or the second retentate enriched for the immunoglobulins may be streamed back into the second tank.

It will be appreciated that the solution enriched in immunoglobulins may be subjected to further purification, using standard techniques, as further described herein.

In a preferred embodiment, there is provided a method for the purification of immunoglobulins, the method comprising:
contacting a sample of blood-derived plasma with a medium chain fatty acid, in conditions comprising: a pH range of between about 4.6 to about 5.0, and conductivity of about 8 mS/cm to about 12 mS/cm, for enabling the selective precipitation of albumin from the sample,
thereby forming a suspension comprising a soluble protein-containing component comprising immunoglobulins and an insoluble protein-containing component comprising albumin;
separating the soluble protein-containing component from the insoluble protein-containing component to obtain a solution of purified immunoglobulins
wherein optionally, the step of separating the soluble protein-containing component from the insoluble protein-containing component comprises feeding the suspension into a filtration unit comprising a dynamic filter element adapted to produce a first retentate and a first filtrate and recovering the first filtrate.

The pH of the sample of blood-derived plasma may be adjusted directly, and without substantial dilution of the sample, for example, with the addition of concentrated acid (such as acetic acid) or a combination of an acid and a base (e.g., NaOH), if required to further adjust the pH. Similarly, the conductivity of the blood-derived plasma may be adjusted directly, without substantial dilution of the sample.

In a third aspect there is provided a method for the purification of immunoglobulins from plasma, the method comprising:
a) in a first tank: mixing a sample of blood-derived plasma with a medium chain fatty acid, in conditions comprising a pH range of between about 4.6 to about 5.0, and optionally, a conductivity of between about 8 mS/cm to about 12 mS/cm, for enabling the selective precipitation of albumin from the sample, thereby forming a suspension comprising soluble immunoglobulins and insoluble albumin;
b) feeding the suspension into a first filtration unit comprising a dynamic filter element adapted to produce a first retentate comprising the insoluble albumin and a first filtrate comprising the soluble immunoglobulin;
c) optionally, diluting the suspension in the first tank by streaming the first retentate into the first tank;
d) recovering the first filtrate in a second tank and,
e) optionally concentrating the first filtrate.

Accordingly, in a fourth aspect, the present invention provides a method for obtaining a solution of immunoglobulins and a precipitate of albumin, the method comprising:
contacting a sample of blood-derived plasma with a medium chain fatty acid, in conditions for enabling the selective precipitation of albumin from the sample,
wherein the conditions comprise a pH range of between about 4.2 to about 5.0;
thereby forming a solution of immunoglobulins and a precipitate of albumin.

Preferably, the conditions also comprise a conductivity of about 5 mS/cm to about 12 mS/cm, preferably 8 mS/cm to about 12 mS/cm. More preferably, if the blood-derived plasma is diluted then the conductivity is equal to or greater than 5 mS/cm but less than, or less than about, 12 mS/cm. If the blood-derived plasma is not diluted then the conductivity is equal to or greater than 8 mS/cm but less than, or less than about, 12 mS/cm.

In a fifth aspect, the present invention provides a method for the purification of immunoglobulins and albumin, the method comprising:
contacting a sample of blood-derived plasma with a medium chain fatty acid, in conditions comprising a pH range of between about 4.2 to about 5.0, for enabling the selective precipitation of albumin from the sample,
thereby forming a suspension comprising a soluble protein-containing component comprising immunoglobulins and an insoluble protein-containing component comprising albumin;
separating the soluble protein-containing component from the insoluble protein-containing component to obtain a solution of purified immunoglobulins and a suspension comprising albumin.

The step of separating the soluble protein-containing component from the insoluble protein-containing component preferably comprises subjecting the suspension to continuous extraction filtration. Preferably, the step of separating the soluble protein-containing component from the insoluble protein-containing component comprises:
feeding the suspension into a filtration unit comprising a dynamic filter element adapted to produce a first retentate and a first filtrate;
recovering the first filtrate and the first retentate.

The first retentate comprising the insoluble protein-containing component will typically be enriched for albumin and the first filtrate comprising the soluble protein-containing component will be enriched for immunoglobulins.

The first filtrate enriched in immunoglobulin may be subjected to a concentration step prior to further processing. The concentration step may comprise a continuous concentration process whereby the first filtrate is fed into a second filtration unit comprising a cross flow filter element or TFF, adapted to produce a second retentate enriched in immunoglobulins; and a second filtrate depleted of immunoglobulins. Optionally, the second filtrate depleted of immunoglobulins may be streamed back into the first tank and/or the second retentate enriched for the immunoglobulins may be streamed back into the second tank.

It will be appreciated that the solution enriched in immunoglobulins may be subjected to further purification, using standard techniques, as further described herein.

In a preferred embodiment, there is provided a method for the purification of immunoglobulins and albumin, the method comprising:
contacting a sample of blood-derived plasma with a medium chain fatty acid, in conditions comprising: a pH range of between about 4.2 to about 5.0, and conductivity of about 8 mS/cm to about 12 mS/cm, for enabling the selective precipitation of albumin from the sample,
thereby forming a suspension comprising a soluble protein-containing component comprising immunoglobulins and an insoluble protein-containing component comprising albumin;
separating the soluble protein-containing component from the insoluble protein-containing component to obtain a solution of purified immunoglobulins and a suspension comprising albumin;
wherein optionally, the step of separating the soluble protein-containing component from the insoluble protein-containing component comprises feeding the suspension into a filtration unit comprising a dynamic filter element adapted to produce a first retentate and a first filtrate and recovering the first filtrate.

The pH of the sample of blood-derived plasma may be adjusted directly, and without substantial dilution of the sample, for example, with the addition of concentrated acid (such as acetic acid) or a combination of an acid and a base (e.g., NaOH), if required to further adjust the pH. Similarly, the conductivity of the blood-derived plasma may be adjusted directly, without substantial dilution of the sample.

In a sixth aspect there is provided a method for the purification of immunoglobulins and albumin from plasma, the method comprising:
a) in a first tank: mixing a sample of blood-derived plasma with a medium chain fatty acid, in conditions comprising a pH range of between about 4.2 to about 5.0, and optionally, a conductivity of between about 8 mS/cm to about 12 mS/cm, for enabling the selective precipitation of albumin from the sample, thereby forming a suspension comprising soluble immunoglobulins and insoluble albumin;
b) feeding the suspension into a first filtration unit comprising a dynamic filter element adapted to produce a first retentate comprising the insoluble albumin and a first filtrate comprising the soluble immunoglobulin;
c) optionally, diluting the suspension in the first tank by streaming the first retentate into the first tank;
d) recovering the first filtrate in a second tank and,
e) optionally concentrating the first filtrate.

Accordingly, in a seventh aspect, the present invention provides a method for obtaining a precipitate of albumin, the method comprising:
contacting a sample of blood-derived plasma with a medium chain fatty acid, in conditions for enabling the selective precipitation of albumin from the sample,
wherein the conditions comprise a pH range of between about 4.15 to about 4.25;
thereby a precipitate of albumin.

Preferably, the conditions also comprise a conductivity of about 5 mS/cm to about 12 mS/cm, preferably 8 mS/cm to about 12 mS/cm. More preferably, if the blood-derived plasma is diluted then the conductivity is equal to or greater than 5 mS/cm but less than, or less than about, 12 mS/cm. If the blood-derived plasma is not diluted then the conductivity is equal to or greater than 8 mS/cm but less than, or less than about, 12 mS/cm.

In an eighth aspect, the present invention provides a method for the purification albumin, the method comprising:
contacting a sample of blood-derived plasma with a medium chain fatty acid, in conditions comprising a pH range of between about 4.15 to about 4.25, for enabling the selective precipitation of albumin from the sample,
thereby forming a suspension comprising a soluble protein-containing component comprising immunoglobulins and an insoluble protein-containing component comprising albumin;
separating the soluble protein-containing component from the insoluble protein-containing component to obtain a precipitate or suspension comprising albumin.

The step of separating the soluble protein-containing component from the insoluble protein-containing component preferably comprises subjecting the suspension to continuous extraction filtration. Preferably, the step of separating the soluble protein-containing component from the insoluble protein-containing component comprises:
feeding the suspension into a filtration unit comprising a dynamic filter element adapted to produce a first retentate and a first filtrate;
recovering the first retentate.

The first retentate comprising the insoluble protein-containing component will typically be enriched for albumin and the first filtrate comprising the soluble protein-containing component will be enriched for immunoglobulins.

It will be appreciated that the first retentate enriched for albumin may be subjected to further purification, using standard techniques, as further described herein.

In a preferred embodiment, there is provided a method for the purification of albumin, the method comprising:
contacting a sample of blood-derived plasma with a medium chain fatty acid, in conditions comprising: a pH range of between about 4.15 to about 4.25, and conductivity of about 8 mS/cm to about 12 mS/cm, for enabling the selective precipitation of albumin from the sample,
thereby forming a suspension comprising a soluble protein-containing component comprising immunoglobulins and an insoluble protein-containing component comprising albumin;
separating the soluble protein-containing component from the insoluble protein-containing component to obtain a suspension comprising albumin;
wherein optionally, the step of separating the soluble protein-containing component from the insoluble protein-containing component comprises feeding the suspension into a filtration unit comprising a dynamic filter element adapted to produce a first retentate and a first filtrate and recovering the first retentate.

The pH of the sample of blood-derived plasma may be adjusted directly, and without substantial dilution of the sample, for example, with the addition of concentrated acid (such as acetic acid) or a combination of an acid and a base (e.g., NaOH), if required to further adjust the pH. Similarly, the conductivity of the blood-derived plasma may be adjusted directly, without substantial dilution of the sample.

In a ninth aspect there is provided a method for the purification of albumin from plasma, the method comprising:
a) in a first tank: mixing a sample of blood-derived plasma with a medium chain fatty acid, in conditions comprising a pH range of between about 4.15 to about 4.25, and optionally, a conductivity of between about 8 mS/cm to about 12 mS/cm, for enabling the selective precipitation of albumin from the sample, thereby forming a suspension comprising soluble immunoglobulins and insoluble albumin;
b) feeding the suspension into a first filtration unit comprising a dynamic filter element adapted to produce a first retentate comprising the insoluble albumin and a first filtrate comprising the soluble immunoglobulin;
c) optionally, diluting the suspension in the first tank by streaming the first retentate into the first tank;
d) recovering the first retentate,

In any aspect, the residual suspension or first retentate in the first tank contains insoluble albumin complexed to the medium chain fatty acid. In order to disrupt binding between albumin and the medium chain fatty acid, the pH of the residual suspension or first retentate may be adjusted to 6.4-6.7, preferably 6.8-7.2. In one embodiment, the pH may be adjusted with 1M sodium hydroxide. Optionally, the first retentate is then subjected to mixing until the pH of the solubilized albumin is stable (typically about 30-60 min). The solution may then be fed into a further filtration unit (e.g. a second system that includes a further filtration unit, e.g. a further filtration unit equivalent to 5 as shown in FIG. 16) comprising a dynamic filter element to produce a retentate depleted of albumin and a filtrate enriched in albumin.

In one embodiment, the filtrate enriched in albumin is then continuously concentrated up to at, or about, 20-45 g/L protein, preferably using a TFF membrane, to form a concentrated albumin solution (e.g. a second system that includes a further filtration unit, e.g. a further filtration unit equivalent to 8 as shown in FIG. 16).

The concentrated albumin solution may then be heated in the range of 60 to 65° C., typically for a time period over 90 min. Without being bound by any theory, it is believed that various proteins other than albumin are denatured at this stage.

After heating the concentrated albumin solution, the pH of the solution may be adjusted to, or about, 4.20, typically with 1 M Hydrochloric acid. At the same time the concentrated albumin solution may be cooled to 4° C. whereby a precipitate is formed of the proteins denatured during the above mentioned heating step. The precipitate may then be removed by filtration whereby the filtrate comprises purified albumin (typically, with an albumin purity equal to or greater than 95%, 96%, 97%, or 98% total protein, and at an albumin yield of equal to or greater than 85%, 86%, 87%, 88%, 89% or 90%).

In any aspect of the present invention, the dynamic filter element in the first filtration unit adapted to produce a filtrate (permeate) enriched with soluble protein (immunoglobulins) is a dynamic cross flow filter element.

In any aspect of the present invention, the dynamic filter element in the second filtration unit adapted to produce a retentate enriched with immunoglobulins is a dynamic cross flow filter element.

In any aspect of the present invention, the dynamic filter element in the further filtration unit adapted to produce a filtrate enriched in albumin is a dynamic cross flow filter element.

In a preferred embodiment the dynamic cross flow filter element is a rotational cross flow filter element. More preferably the rotational cross flow filter element comprises a filter disc. The filter discs are usually mounted on a shaft member. In an embodiment the rotational cross flow filter element comprises at least one filter disc and at least one shaft member.

According to a preferred embodiment of any aspect of the present invention, the filter disc membrane is a ceramic membrane. More preferably the ceramic membrane has a pore size in the range of greater than or equal to 5 nm to less than or equal to 2 μm. In particular embodiments, the ceramic membrane has a pore size of from about 0.2 μm to 2 μm. In particular embodiments, the ceramic filter membrane has an average pore size in the range of greater than or equal to 5 nm to less than or equal to 200 nm (0.2 μm). In particular embodiments, the ceramic filter membrane has an average pore size in the range of greater than or equal to 50 nm to less than or equal to 100 nm. Such filter discs are supplied by Kerafol and Flowserve.

It will be appreciated that the sample of blood-derived plasma may include any plasma sample derived from blood, preferably human blood. In certain embodiments, the sample of blood-derived plasma comprises fresh plasma, cryo-poor plasma, or cryo-rich plasma. The plasma may be obtained from a number of donations and/or subjects, and pooled. The plasma may be hyperimmune plasma.

Preferably, the sample of plasma does not comprise filter aid and/or has not been subjected to an alcohol or other fractionation processes.

In any of the first, second or third aspects, the pH of the plasma sample is adjusted to a pH range of about 4.6 to about 5.0, prior to the contacting and/or mixing with the medium chain fatty acid. For example, the pH of the plasma sample may be adjusted to a pH of about 4.6, about 4.7, about 4.8, about 4.9, about 5.0. The pH of the plasma sample may be adjusted to a pH of 4.6, a pH of 4.7, a pH of 4.8, a pH of 4.9 or a pH of 5.0.

In any of the fourth, fifth or sixth aspects, the pH of the plasma sample is adjusted to a pH range of about 4.2 to about 5.0, prior to the contacting and/or mixing with the medium chain fatty acid. For example, the pH of the plasma sample may be adjusted to a pH of about 4.2, about 4.3, about 4.4, about 4.5, about 4.6, about 4.7, about 4.8, about 4.9 or about 5.0. The pH of the plasma sample may be adjusted to a pH of 4.2, a pH of 4.3, a pH of 4.4, a pH of 4.5, a pH of 4.6, a pH of 4.7, a pH of 4.8, a pH of 4.9 or a pH of 5.0.

In any of the seventh, eighth or ninth aspects, the pH of the plasma sample is adjusted to a pH range of about 4.15 to about 4.25, prior to the contacting and/or mixing with the medium chain fatty acid. For example, the pH of the plasma sample may be adjusted to a pH of about 4.15, about 4.16, about 4.17, about 4.18, about 4.19. about 4.20, about 4.21, about 4.22, about 4.23, about 4.24 or about 4.25. The pH of the plasma sample may be adjusted to a pH of 4.15, a pH of 4.16, a pH of 4.17, a pH of 4.18, a pH of 4.19, a pH of 4.20, a pH of 4.21, a pH of 4.22, a pH of 4.23, a pH of 4.24 or a pH of 4.25.

In any aspect, the pH of the plasma sample may be adjusted without substantial dilution of the plasma prior to contact with the medium chain fatty acid. Such pH adjustment may be accomplished with the addition of concentrated acid, such as acetic acid, or a combination of an acid and a base (e.g., NaOH), if required to further adjust the pH. If required, the conductivity of the resulting solution is adjusted to achieve the desired conductivity of about 8 mS/cm to about 12 mS/cm.

In certain embodiments of any aspect of the invention, the plasma sample may be diluted in a buffer prior to the step of being mixed with the medium chain fatty acid. The dilution of plasma may be a dilution of about 1:0.5, about 1:0.75, about 1:1, about 1:1.25, about 1:1.5, about 1:1.75 or about 1:2. In circumstances where the plasma is diluted the conductivity may be about 5 mS/cm to about 12 mS/cm.

The buffer in which the plasma is diluted may be any suitable buffer for diluting plasma, for example, an acetate buffer (e.g., sodium acetate). The acetate buffer may comprise sodium acetate trihydrate and glacial acetic acid. The concentration of the buffer may be 60 mM, 80 mM, 100 mM, or 0.22 M, and may have a pH of, or about, 4.1; of, or about, 4.2; of, or about, 4.3; of or about, 4.4; of, or about, 4.5; of, or about, 4.6; of, or about, 4.7; of, or about, 4.8. Alternatively, a phosphate-acetate buffer may be used. The phosphate-acetate buffer may comprise 10 mM phosphate (e.g. sodium phosphate) and 10 mM acetate (e.g. sodium acetate). The pH of this buffer may be about 4.3 to about 4.4. If the conductivity of the resulting solution needs to be adjusted to achieve the desired conductivity of about 5 to about 12 mS/cm, preferably about 8 to about 12 mS/cm, this can be achieved with, for example, addition of a more concentrated acetate buffer (e.g. 3.5 M acetate buffer comprising sodium acetate trihydrate and glacial acetic acid, pH 5).

Typically, the buffer is used not only to dilute the plasma but also to facilitate pH adjustment of the plasma sample. Accordingly, in any embodiment of the first, second or third aspects of the present invention, the pH of the diluted plasma sample is a pH of about 4.6 to about 5.0. For example, the pH of the diluted plasma sample may be a pH of about 4.6, about 4.7, about 4.8, about 4.9, about 5.0. The pH of the diluted plasma sample may be a pH of 4.6, a pH of 4.7, a pH of 4.8, a pH of 4.9 or a pH of 5.0. Accordingly, in any embodiment of the fourth, fifth or sixth aspects of the present invention, the pH of the diluted plasma sample is a pH of about 4.2 to about 5.0. For example, the pH of the diluted plasma sample may be a pH of about 4.2, about 4.3, about 4.4, about 4.5, about 4.6, about 4.7, about 4.8, about 4.9 or about 5.0. The pH of the diluted plasma sample may be a pH of 4.2, a pH of 4.3, a pH of 4.4, a pH of 4.5, a pH of 4.6, a pH of 4.7, a pH of 4.8, a pH of 4.9 or a pH of 5.0. Accordingly, in any embodiment of the seventh, eighth or ninth aspects of the present invention, the pH of the diluted plasma sample is a pH of about 4.15 to about 4.25. For example, the pH of the diluted plasma sample may be a pH of about 4.15, about 4.16, about 4.17, about 4.18, about 4.19. about 4.20, about 4.21, about 4.22, about 4.23, about 4.24 or about 4.25. The pH of the diluted plasma sample may be a pH of 4.15, a pH of 4.16, a pH of 4.17, a pH of 4.18, a pH of 4.19, a pH of 4.20, a pH of 4.21, a pH of 4.22, a pH of 4.23, a pH of 4.24 or a pH of 4.25.

In any aspect or embodiment, the conditions may comprise a conductivity of about 5 mS/cm to about 12 mS/cm, preferably 8 mS/cm to about 12 mS/cm. More preferably, if the blood-derived plasma is diluted then the conductivity is equal to or greater than 5 mS/cm but less than, or less than about, 12 mS/cm. If the blood-derived plasma is not diluted then the conductivity is equal to or greater than 8 mS/cm but less than, or less than about, 12 mS/cm.

For example, the conductivity of the diluted or undiluted plasma sample is about 8 to about 12 mS/cm. As such, in any embodiment, the conductivity of the diluted or undiluted plasma sample is about 8 mS/cm, about 9 mS/cm, about 10 mS/cm, about 11 mS/cm or about 12 mS/cm. In any embodiment, the conductivity of the diluted or undiluted plasma sample is 8 mS/cm, 9 mS/cm, 10 mS/cm, 11 mS/cm or 12 mS/cm. In any embodiment, the conductivity of the diluted plasma is equal to or greater than 5 mS/cm, 6 mS/cm or 7 mS/cm. Typically, conductivity is measured at room temperature, preferably the conductivity is measured between 18 to 25° C.

In any embodiment, the medium chain fatty acid may be selected from a fatty acid that comprises a general structural formula of CH₃(CH₂)ₙCOOH, wherein the fatty acid is a C 4 to C 10 carboxylic acid. The fatty acid may be saturated or unsaturated. More preferably, the fatty acid comprises enanthic (heptanoic) acid, caprylic (octanoic) acid, octenoic acid, pelargonic (nonanoic) acid, nonenoic acid, or capric (decanoic) acid. Most preferably, the fatty acid is caprylic (octanoic) acid. Also contemplated as the reagent is a salt or ester of any fatty acid described herein, e.g. caprylate.

In any embodiment of the first, second and third aspects, the amount of fatty acid, preferably caprylic (octanoic) acid, mixed with the plasma sample, is about 0.30 g/g total protein (in the plasma sample), about 0.35 g/g total protein, about 0.40 g/g total protein, about 0.45 g/g total protein or about 0.50 g/g total protein. Preferably, the amount of fatty acid, preferably caprylic (octanoic) acid, is about 0.300 g/g total protein, or about 0.325 g/g total protein, or about 0.350 g/g total protein, or about 0.375 g/g total protein, or about 0.400 g/g total protein, or about 0.425 g/g total protein, or about 0.450 g/g total protein. More preferably, the amount of fatty acid, preferably caprylic (octanoic) acid, is at least about 0.350 g/g total protein. Preferably, the amount of fatty acid, preferably caprylic (octanoic) acid is in the range from about 0.38 g/g total protein to about 0.50 g/g total protein. More preferably, the amount of fatty acid, preferably caprylic (octanoic) acid, is about 0.38 g/g total protein, about 0.39 g/g total protein, about 0.40 g/g total protein, about 0.41 g/g total protein, about 0.42 g/g total protein, about 0.43 g/g total protein, about 0.44 g/g total protein, about 0.45 g/g total protein, about 0.46 g/g total protein, about 0.47 g/g total protein, about 0.48 g/g total protein, about 0.49 g/g total protein, or about 0.50 g/g total protein.

In any embodiment of the first, second and third aspects, the amount of fatty acid, preferably caprylic (octanoic) acid, is 0.30 g/g total protein (in the plasma sample), 0.35 g/g total protein, 0.40 g/g total protein, 0.45 g/g total protein or 0.50 g/g total protein. Preferably, the amount of fatty acid, preferably caprylic (octanoic) acid, is 0.300 g/g total protein, or 0.325 g/g total protein, or 0.350 g/g total protein, or 0.375 g/g total protein, or 0.400 g/g total protein, or 0.425 g/g total protein, or 0.450 g/g total protein. More preferably, the amount of fatty acid, preferably caprylic (octanoic) acid, is at least 0.350 g/g total protein. Preferably, the amount of fatty acid, preferably caprylic (octanoic) acid is in the range from 0.38 g/g total protein to 0.50 g/g total protein. More preferably, the amount of fatty acid, preferably caprylic (octanoic) acid, is 0.38 g/g total protein, 0.39 g/g total protein, 0.40 g/g total protein, 0.41 g/g total protein, 0.42 g/g total protein, 0.43 g/g total protein, 0.44 g/g total protein, 0.45 g/g total protein, 0.46 g/g total protein, 0.47 g/g total protein, 0.48 g/g total protein, 0.49 g/g total protein, or 0.50 g/g total protein.

In any embodiment of the fourth, fifth and sixth aspects, the amount of fatty acid, preferably caprylic (octanoic) acid, mixed with the plasma sample, is about 0.35 g/g total protein (in the plasma sample), about 0.40 g/g total protein, about 0.45 g/g total protein, about 0.50 g/g total protein or about 0.55 g/g total protein. Preferably, the amount of fatty acid, preferably caprylic (octanoic) acid, is about 0.35 g/g total protein, about 0.36 g/g total protein, 0.37 g/g total protein, 0.38 g/g total protein, about 0.39 g/g total protein, about 0.40 g/g total protein, about 0.41 g/g total protein, about 0.42 g/g total protein, about 0.43 g/g total protein, about 0.44 g/g total protein, about 0.45 g/g total protein, about 0.46 g/g total protein, about 0.47 g/g total protein, about 0.48 g/g total protein, about 0.49 g/g total protein, about 0.50 g/g total protein, about 0.51 g/g total protein, about 0.52 g/g total protein, about 0.53 g/g total protein, about 0.54 g/g total protein, or about 0.55 g/g total protein.

In any embodiment of the fourth, fifth and sixth aspects, the amount of fatty acid, preferably caprylic (octanoic) acid, mixed with the plasma sample, is 0.36 g/g/total protein, 0.37 g/g total protein, 0.38 g/g total protein, 0.39 g/g total protein, 0.40 g/g total protein, 0.41 g/g total protein, 0.42 g/g total protein, 0.43 g/g total protein, 0.44 g/g total protein, 0.45 g/g total protein, 0.46 g/g total protein, 0.47 g/g total protein, 0.48 g/g total protein, 0.49 g/g total protein, 0.50 g/g total protein, 0.51 g/g total protein, 0.52 g/g total protein, 0.53 g/g total protein, 0.54 g/g total protein, or 0.55 g/g total protein.

In any embodiment of the seventh, eighth or ninth aspects, the amount of fatty acid, preferably caprylic (octanoic) acid, mixed with the plasma sample, is equal to or greater than about 0.35 g/g total protein. Preferably, the amount of fatty acid, preferably caprylic (octanoic) acid, mixed with the plasma sample, is equal to or greater than about 0.35 g/g total protein but less than, or less than about, 1.1 g/g total protein. The amount of fatty acid, preferably caprylic (octanoic) acid, mixed with the plasma sample may be about 0.35 g/g total protein, about 0.36 g/g total protein, about 0.37 g/g total protein, about 0.38 g/g total protein, about 0.39 g/g total protein, about 0.40 g/g total protein, about 0.41 g/g total protein, about 0.42 g/g total protein, about 0.43 g/g total protein, about 0.44 g/g total protein, about 0.45 g/g total protein, about 0.46 g/g total protein, about 0.47 g/g total protein, about 0.48 g/g total protein, about 0.49 g/g total protein, about 0.50 g/g total protein, about 0.51 g/g total protein, about 0.52 g/g total protein, about 0.53 g/g total protein, about 0.54 g/g total protein, about 0.55 g/g total protein, about 0.56 g/g total protein, about 0.57 g/g total protein, about 0.58 g/g total protein, about 0.59 g/g total protein, about 0.60 g/g total protein, about 0.61 g/g total protein, about 0.62 g/g total protein, about 0.63 g/g total protein, about 0.64 g/g total protein, about 0.65 g/g total protein, about 0.66 g/g total protein, about 0.67 g/g total protein, about 0.68 g/g total protein, about 0.69 g/g total protein, about 0.70 g/g total protein, about 0.71 g/g total protein, about 0.72 g/g total protein, about 0.73 g/g total protein, about 0.74 g/g total protein, about 0.75 g/g total protein, about 0.76 g/g total protein, about 0.77 g/g total protein, about 0.78 g/g total protein, about 0.79 g/g total protein, about 0.80 g/g total protein, about 0.81 g/g total protein, about 0.82 g/g total protein, about 0.83 g/g total protein, about 0.84 g/g total protein, about 0.85 g/g total protein, about 0.86 g/g total protein, about 0.87 g/g total protein, about 0.88 g/g total protein, about 0.89 g/g total protein, about 0.90 g/g total protein, about 0.91 g/g total protein, about 0.92 g/g total protein, about 0.93 g/g total protein, about 0.94 g/g total protein, about 0.95 g/g total protein, about 0.96 g/g total protein, about 0.97 g/g total protein, about 0.98 g/g total protein, about 0.99 g/g total protein, about 1.0 g/g total protein, about 1.01 g/g total protein, about 1.02 g/g total protein, about 1.03 g/g total protein, about 1.04 g/g total protein, about 1.05 g/g total protein, about 1.06 g/g total protein, about 1.07 g/g total protein, about 1.08 g/g total protein, about 1.09 g/g total protein, or about 1.1 g/g total protein.

In any embodiment of the fourth, fifth and sixth aspects, the amount of fatty acid, preferably caprylic (octanoic) acid, mixed with the plasma sample, is 0.35 g/g total protein, 0.36 g/g total protein, 0.37 g/g total protein, 0.38 g/g total protein, 0.39 g/g total protein, 0.40 g/g total protein, 0.41 g/g total protein, 0.42 g/g total protein, 0.43 g/g total protein, 0.44 g/g total protein, 0.45 g/g total protein, 0.46 g/g total protein, 0.47 g/g total protein, 0.48 g/g total protein, 0.49 g/g total protein, 0.50 g/g total protein, 0.51 g/g total protein, 0.52 g/g total protein, 0.53 g/g total protein, 0.54 g/g total protein, 0.55 g/g total protein, 0.56 g/g total protein, 0.57 g/g total protein, 0.58 g/g total protein, 0.59 g/g total protein, 0.60 g/g total protein, 0.61 g/g total protein, 0.62 g/g total protein, 0.63 g/g total protein, 0.64 g/g total protein, 0.65 g/g total protein, 0.66 g/g total protein, 0.67 g/g total protein, 0.68 g/g total protein, 0.69 g/g total protein, 0.70 g/g total protein, 0.71 g/g total protein, 0.72 g/g total protein, 0.73 g/g total protein, 0.74 g/g total protein, 0.75 g/g total protein, 0.76 g/g total protein, 0.77 g/g total protein, 0.78 g/g total protein, 0.79 g/g total protein, 0.80 g/g total protein, 0.81 g/g total protein, 0.82 g/g total protein, 0.83 g/g total protein, 0.84 g/g total protein, 0.85 g/g total protein, 0.86 g/g total protein, 0.87 g/g total protein, 0.88 g/g total protein, 0.89 g/g total protein, 0.90 g/g total protein, 0.91 g/g total protein, 0.92 g/g total protein, 0.93 g/g total protein, 0.94 g/g total protein, 0.95 g/g total protein, 0.96 g/g total protein, 0.97 g/g total protein, 0.98 g/g total protein, 0.99 g/g total protein, 1.0 g/g total protein, 1.01 g/g total protein, 1.02 g/g total protein, 1.03 g/g total protein, 1.04 g/g total protein, 1.05 g/g total protein, 1.06 g/g total protein, 1.07 g/g total protein, 1.08 g/g total protein, 1.09 g/g total protein, or 1.1 g/g total protein.

In preferred embodiments, the step of contacting the plasma sample with the medium chain fatty acid comprises mixing of the plasma sample and fatty acid to obtain a homogeneous emulsion of the medium chain fatty acid and plasma sample. In preferred embodiments, the mixing is vigorous mixing, so as to enable formation of the homogeneous emulsion.

Preferably, the plasma sample and the medium chain fatty acid, preferably caprylic (octanoic) acid, are mixed for a period of at least about 10 minutes, at least about 15 minutes, at least about 20 minutes, at least about 25 minutes, at least about 30 minutes, at least about 35 minutes, at least about 40 minutes, at least about 45 minutes, or at least about 50 minutes or more.

Preferably, the mixing step is followed by a period of incubation, prior to the step of separating the soluble protein-containing component (soluble immunoglobulins) from the insoluble protein-containing component (insoluble albumin). The period of incubation is preferably at least about 20 minutes, at least about 30 minutes, at least about 40 minutes, at least about 50 minutes, at least about 60 minutes, at least about 70 minutes, at least about 80 minutes, at least about 90 minutes, at least about 100 minutes, at least about 110 minutes, at least about 120 minutes, at least about 130 minutes, at least about 140 minutes, at least about 150 minutes or more.

In any embodiment of any aspect of the invention, unless otherwise specified, the steps of the method are performed at a temperature of between about 18° C. to about 37° C., preferably between about 18° C. to about 24° C. In any embodiment, the temperature is about 18° C., about 19° C., about 20° C., about 21° C., about 22° C., about 23° C., or about 24° C. In any embodiment, the temperature is 18° C., 19° C., 20° C., 21° C., 22° C., 23° C., or 24° C.

The immunoglobulins purified according to any aspect of the invention, preferably comprise immunoglobulin G (IgG), preferably human immunoglobulin G (IgG). The immunoglobulins may comprise any one of the IgG subclasses IgG1, IgG2, IgG3 or IgG4, preferably wherein the relative distribution of the IgG subclasses is similar, or substantially the same as the distribution of IgG subclasses typically observed in plasma. Optionally, the IgG1 is present in the composition in an amount of about 60% to about 70% of total immunoglobulin, IgG2 is present at about 25% to about 35% of total immunoglobulin, IgG3 is present at about 2% to about 3% of total immunoglobulin and IgG4 is present at about 0.5% to about 1.5% of total immunoglobulin.

In any embodiment of any aspect of the invention, the immunoglobulin solution, or concentrated immunoglobulin is subjected to further processing in order to further purify the immunoglobulin. Preferably, the further processing does not comprise a further step of continuous filter extraction.

In certain embodiments, the immunoglobulin is subjected to further processing such as low pH treatment, chromatography steps (including anion exchange chromatography and/or immunoaffinity chromatography), virus filtration and inactivation steps, concentration and formulation so that the end product can be administered for example to the human body. The end product can be used in the treatment of immune conditions, particular autoimmune diseases and certain neurological diseases. These conditions include Rheumatoid arthritis, Systemic Lupus Erythematosus (SLE), Antiphospholipid syndrome, immune thrombocytopenia (ITP), Kawasaki disease, Guillain Barre syndrome (GBS), multiple sclerosis (MS), chronic inflammatory demyelinating polyneuropathy (CIDP), multifocal motor neuropathy (MMN), myasthenia gravis (MG), skin blistering diseases, scleroderma, Dermatomyositis, Polymyositis, Alzheimer's Disease, Parkinson's Disease, Alzheimer's Disease related to Downs Syndrome, cerebral amyloid angiopathy, Dementia with Lewy bodies, Fronto-temporal lobar degeneration or vascular dementia. In addition, the end IVIg and SCIg products can be used in other medical procedures such as in cell and organ transplant.

In any embodiment of any aspect of the invention, the purified immunoglobulin solution contains one or more of the following impurities: IgA, IgM, albumin, α (alpha)-2 macroglobulin, α (alpha)-1 anti-trypsin, a lipid, and a lipoprotein.

In any aspect, the insoluble protein-containing component of the suspension (i.e., obtained from contacting the plasma sample with the medium chain fatty acid), is retained following the step of separating the soluble protein-containing component from the insoluble protein-containing component.

It will be appreciated that the insoluble protein-containing component, can then be used for the purposes of obtaining a purified fraction of albumin, for example as described herein.

For example, in the context of the third, sixth or ninth aspects of the invention, once the suspension obtained from mixing the plasma and medium chain fatty acid has been fed through the first filtration unit, any residual suspension, and/or the first retentate, can be further processed to obtain purified albumin.

Accordingly, in any aspect of the invention, the method further comprises:
a) adjusting the pH of the insoluble protein-containing component comprising albumin to a pH of between about 6.4 to about 7.2 (preferably about 6.8 to about 7.2), to obtain solubilised albumin;
b) optionally, subjecting the solubilised albumin to further processing steps to remove impurities therefrom;
c) recovering purified albumin from the solubilised albumin.

In the context of the third, sixth and ninth aspects of the invention, the insoluble albumin may be residual insoluble protein remaining in the first tank and/or may further comprise the first retentate.

The adjusting of the pH of the insoluble protein-containing component may be carried out directly, without substantial dilution of the insoluble protein-containing component. Such pH adjustment may be accomplished with the addition of concentrated acid, such as acetic acid, or a combination of an acid and a base (e.g., NaOH), if required to further adjust the pH. For example, the insoluble-protein containing component (e.g. residual suspension in the first tank) may have a pH adjustment to 6.4 to 7.2 (preferably 6.8 to 7.2) with 1M sodium hydroxide or phosphate buffer (pH 7.1-7.4). Also contemplated is a combination of sodium hydroxide and phosphate buffer at 0.12M. If required, the conductivity of the resulting solution is adjusted to achieve the desired conductivity of about 8 mS/cm to about 15 mS/cm.

Optionally, the adjusting the pH of the insoluble protein-containing component comprises first contacting the insoluble protein-containing component (insoluble albumin) with a buffer, having a pH of between 7.1 to 7.4, and optionally a conductivity of between about 8 to about 15 mS/cm, to form a further suspension, and adjusting the pH of this further suspension to a pH of at least about 6.4, preferably neutral, more preferably between about 6.4 to about 7.2, or about 6.4 to about 6.7, or about 6.8 to about 7.2, thereby obtaining solubilised albumin.

Any buffers that can disrupt binding between albumin and the medium chain fatty acid, so as to liberate the fatty acid from the albumin thereby solubilising the albumin, are suitable for use in this step. For example, the buffer may have a pH around 7 and conductivity of about 8 to about 15 mS/cm. The step may be carried out by adding the buffer to the tank containing the suspension, and stirring (for, for example, 5 minutes) until the pH changes to about 7, preferably about 7.2 (in particular, to at least about 6.4, more preferably between about 6.4 to about 6.7. about 6.4 to about 7.2, most preferably about 6.8 to about 7.2). An example of a suitable buffer is a phosphate buffer. The phosphate buffer may comprise NaH₂PO₄.2H₂O and NaH₂PO₄.12H₂O. The buffer may have a concentration of 0.12 M, pH 7.3±0.2. Alternatively, the pH adjustment may be carried out directly using a base, such as NaOH, as described above.

The solubilised albumin may be subjected to further processing steps to remove impurities.

In one embodiment of any aspect of the invention, the further processing of the solubilised albumin comprises:
feeding the solubilised albumin into a filtration unit comprising a dynamic filter element adapted to produce a retentate depleted of albumin and a filtrate enriched in albumin;
recovering the filtrate enriched with albumin.

The filtrate enriched in albumin may optionally be subjected to a concentration step prior to further processing. The concentration step may comprise a continuous concentration process whereby the filtrate is fed into a second filtration unit comprising a cross flow filter element adapted to produce a retentate enriched in albumin; and a filtrate depleted of albumin.

In accordance with this embodiment of the invention, the method for further processing of the solubilised albumin may therefore comprise:
d) providing the solubilised albumin in a first tank:
e) feeding the solution of solubilised albumin into a first filtration unit comprising a dynamic filter element adapted to produce a retentate depleted of albumin and a filtrate enriched in soluble albumin;
f) optionally, diluting the solution in the first tank by streaming the retentate into the first tank;
g) recovering the filtrate, enriched in albumin, in a second tank and,
h) optionally concentrating the filtrate.

In any embodiment, the step h) of concentrating the filtrate comprises subjecting the filtrate to a continuous concentration process in a second filtration unit comprising a dynamic filter element or TFF adapted to produce a retentate enriched for albumin and a filtrate depleted of albumin. Optionally, the filtrate depleted of albumin may be streamed back into the first tank and/or the retentate enriched for the albumin may be streamed back into the second tank.

Preferably, the dynamic filter element in the first filtration unit adapted to produce a filtrate (permeate) enriched with albumin is a dynamic cross flow filter element. Preferably, the dynamic filter element in the second filtration unit adapted to concentrate the albumin solution, and produce a retentate enriched with albumin, is a dynamic cross flow filter element or TFF.

In alternative embodiments, the solubilised albumin fraction may be subjected to alcohol precipitation methods and/or chromatography as is generally known in the art, for further purifying the albumin. Depending on the nature of the contaminants present in the albumin, various purification schemes could be adopted. For example, the albumin could be subjected to well-known fractionation processes, such as ethanol fractionation to produce Supernatant I, Supernatant II+III, Supernatant-IV-1, Supernatant-IV-4, or Fraction V. The albumin may be further pH-adjusted, ultrafiltered/diafiltered, and pasteurized, as per the AlbuRx production process. Other methods for producing purified albumin are well-known, and include subjecting the albumin to ion exchange chromatography, followed by gel filtration chromatography and pasteurization, as conducted in the Albumex production process. Suitable albumin purification processes are discussed in Matejtschuk, P. et al (2000) British Journal of Anaesthesia 85 (6); 887-95, and in the Australian Public Assessment Report for Albumin (human) (2017) Therapeutic Goods Administration, pages 8-9 (available online at: https://www.tga.gov.au/sites/default/files/auspar-albumin-human-170502.pdf).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

The following drawings are not necessarily drawn to scale, emphasis instead is generally being placed upon illustrating the principles of various embodiments. In the following description, various embodiments of the invention are described with reference to the following drawings:

FIG. 1: Distribution of IgG subclasses in blood-derived plasma and in clarified filtrate comprising purified immunoglobulins obtained according to the methods of the invention.

FIG. 2: Protease composition of blood-derived plasma; diluted pooled plasma; incubation with octanoic acid at 0 hours, 1 hour, 2 hours and 3 hours (T0, T1, T2 and T3, respectively); and UF/DF filtrate comprising purified immunoglobulins prepared according to the methods of the invention. Protease levels are given as nkat/L.

FIG. 3: Impurities detected in plasma and clarified filtrate comprising purified immunoglobulins obtained according to the methods of the invention.

FIG. 4: Activity (IU/mL) of Prekallikrein activator (PKA), Factor IX (FIX) and Factor XI(a) (FXI(a)), in plasma, and in clarified filtrate comprising purified immunoglobulins obtained according to the methods of the invention.

FIG. 5: Concentration of albumin (g/L) in blood-derived plasma and a clarified filtrate comprising purified immunoglobulins obtained according to the methods of the invention.

FIG. 6: Protein composition of plasma and of clarified filtrate comprising purified immunoglobulins obtained according to the methods of the invention. Relative percentage of gamma-globulin, alpha-/beta-globulin and albumin are shown.

FIG. 7: IgG yield (g/L plasma) at different octanoic acid amount and different pH at constant ionic strength.

FIG. 8: Albumin yield (g/L plasma) at different octanoic acid amount and different pH at constant ionic strength.

FIG. 9: IgG, albumin, IgA and IgM yield (g/L plasma) at constant pH at varying ionic strength.

FIG. 10: IgG yield (g/L plasma) of clarified and concentrated octanoic acid filtrate at various pH and various ionic strength.

FIG. 11: Albumin yield (g/L plasma) of clarified and concentrated octanoic acid filtrate at various pH and various ionic strength.

FIG. 12: IgA yield (g/L plasma) of clarified and concentrated octanoic acid filtrate at various pH and various ionic strength.

FIG. 13: IgM yield (g/L plasma) of clarified and concentrated octanoic acid filtrate at various pH and various ionic strength.

FIG. 14: Product related impurities (g/L plasma).

FIG. 15: Impurity profile (g/L plasma) of heat treated albumin.

FIG. 16: Schematic flow chart overview of a continuous extraction filtration system.

FIG. 16 illustrates a schematic flow chart overview of the system 100 and the method according to one preferred embodiment of the present invention. Plasma is placed in Tank 1, pH and conductivity are adjusted by adding buffer/acid etc. After that the OA is added and the OA suspension is incubated in Tank 1. The OA suspension can be fed to a first filtration unit 5, through the pump 2, several type of pumps can be used (e.g. piston-; rotary-; centrifugal- and membrane pump) and flow-regulated valve 3 of a pipe 12. The first filtration unit 5 is equipped with a rotating hollow shaft to which the filter discs are mounted (the filtrate flows from the outside to the inside of hollow shaft). The first filtration unit 5 is further set up with height adjustable scrapers to keep the filter cake thickness constant and thus achieve constant filtrate flow. The desired filtration pressure is controlled and regulated by overflow valve (unfiltered suspension outlet). The filter discs used can be a ceramic membrane, depth filter layers and sintered porous metal filter discs. Once the vessel of the first filtration unit 5 is filled with the suspension, a continuous pressure extraction and separation can be started. The first filtration unit 5, which can comprise a pressure unit/vessel, is provided with suitable internal settings and conditions to simultaneously increase the extraction efficiency and filtration process. The extraction efficiency is increased through turbulence mixing in the unit 5 without having to involve a mixer. Nevertheless, it can be foreseen that an additional mixer may be provided to assist the extraction process by creating turbulences. Moreover, higher final dilution factor e.g. 1: ≥30 disclosed in the present invention also increases the extraction efficiency, leading to high protein (e.g. IgG) yield. Of course, any other higher final dilution factor (higher than 70) can also be envisaged.

The first filtrate flows through a flowmeter 6 installed on pipe (or channel) 14 and is collected in the second tank 7. The unfiltered suspension (e.g. first retentate) flows back through the regulated outlet 3 installed on pipe 13 in tank 1. When a defined volume in the second tank 7 is reached, the UF 8 concentration process can be started in the second filtration unit. The first filtrate in the second tank 7 flows through pipe 15 into the ultrafiltration (UF) system 8 (e.g. Tangential Flow Filtration (TFF) using a TFF membrane). The transmembrane pressure is set such that the permeate flow rate 17 is identical or almost identical to that with the first filtrate flow rate in pipe 14. The permeate (or second filtrate) of the UF system 8 flows through pipe (or line or channel) 17 back to the first tank 1, whereas the retentate (or second retentate) of the UF 8 system (=concentrated protein) flows through pipe 16 back to the second tank 7.

In accordance with the invention, the first process unit 5 is provided with one or more rotating filter discs comprising one or more of the first filter element for turbulence mixing of the content of the first process unit 5 for producing the first retentate and the first permeate/filtrate. The first retentate can be fed back to the first tank 1 through a channel 13 via a control valve 3 whereas the first permeate/filtrate can be fed to a second tank 7 via another channel 14. The first filter element can be a filtration membrane which is based on a ceramic material, having a pore diameter of between about 5 nm to 5000 nm, preferably between 20 nm to 100 nm or more preferably between 30 nm to 80 nm. It can also be foreseen that inorganic membranes or any other suitable membranes could also provide a similar effect as the ceramic based membrane. The first filtration unit 5 may be supplied with a pressure control device 4 such as a manometer in order to regulate the pressure within. Similarly, a flowmeter 6 can be installed in the system of the present invention for measuring the flow rate of the suspension or solution.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

The present invention relates to a system and a method for efficiently purifying immunoglobulin, preferably IgG, from plasma. The invention advantageously enables the use of plasma as the starting material, and without the need for alcohol-based precipitation methods, to enable the extraction of immunoglobulin from plasma in a single step.

A further advantage of the invention is the ability to simultaneously purify albumin from the starting plasma sample, thereby providing a method for rapidly obtaining substantially pure preparations of both immunoglobulin and albumin from plasma via a single precipitation and filtration step.

A particular benefit of the approach of the methods of the invention is the absence of any filter aid, which advantageously ensures that protease activity is reduced early on in the process, ensuring maximum protein recovery and yield. Further advantages arise, in part, from the application of a single continuous extraction filtration method to separate the immunoglobulin-containing component and albumin-containing components of plasma. The continuous extraction method facilitates downstream processing using conditions which minimize loss of the protein of interest, for example, by reducing the total amount of reagent required to precipitate impurities or proteins that are not of interest.

### Definitions

The term “soluble protein-containing component” is intended to refer to the water-soluble component or components of the aqueous phase that result following mixing of a medium chain fatty acid with a sample of blood-derived plasma, according to the methods of the invention. Typically, the soluble protein-containing component is highly enriched for immunoglobulins and other proteins that remain soluble following mixing of the plasma with the fatty acid.

The term “insoluble protein-containing component” is intended to refer to the water-insoluble component or components of the solid phase that result following mixing of a medium chain fatty acid with a sample of blood-derived plasma, according to the methods of the invention. Typically, the insoluble protein-containing component comprises precipitated proteins, principally albumin, but also other contaminating proteins which are denatured following mixing of the plasma with the fatty acid.

The methods of the invention enable the selective precipitation of albumin from a sample of blood-derived plasma. In preferred embodiments, the precipitation results in at least 50% of the albumin in the sample being precipitated. More preferably, at least about 50%, at least about 55%, at least about 60%, at least about 65%, at least about 70%, at least about 75%, at least about 80%, at least about 85%, or at least about 90%, or more, of the albumin in the sample is precipitated.

By “high yield” it is meant that the yield of the protein of interest such as immunoglobulin G or albumin (as well as other proteins and immunoglobulins) is at least 80%, at least 85%, at least 95% of the amount of the protein in the soluble or insoluble protein-containing component, preferably at least 96%, more preferably at least 98%, most preferably more than 98%.

The concentration of immunoglobulin and/or albumin in a sample can be measured by any means known to persons skilled in the art. It will be understood that the method used to measure immunoglobulins or albumin may depend on the nature of the sample. For example, it will be understood that, where the sample is an albumin-containing precipitate, it may be necessary to dissolve the precipitate (or a sample thereof) in a suitable buffer prior to the measurement. Examples of suitable assays for measuring a protein of interest include high pressure liquid chromatography (HPLC; e.g., size exclusion HPLC), enzyme-linked immunosorbent assay (ELISA) and quantitative immunonephelometry.

The pH and/or conductivity of any sample can be measured by known methods in the art. Typically, pH and/or conductivity are measured at room temperature, preferably the pH and/or conductivity are measured between 18 to 25° C. The unit of measurement for conductivity is milliSiemens per centimeter (mS/cm), and can be measured using a standard conductivity meter. The conductivity of a solution can be altered by changing the concentration of ions therein. For example, the concentration of a buffering agent and/or concentration of a salt (e.g. NaCl or KCl) in the solution may be altered in order to achieve the desired conductivity. Preferably, the salt concentration is modified to achieve the desired conductivity as described in the Examples below or elsewhere herein.

By “about” or “approximately” in relation to a given numerical value for percentage, pH, amount or a period of time or other references, it is meant to include numerical values within 10% of the specified value.

### Large or Industrial Scale Processes or Systems

Large or industrial scale with regard to the present invention represents production procedures based on at least 200 L, preferably at least 500 L, even more preferably at least 2000 L of a starting material such as human plasma. For example typical commercial plasma donor pool sizes used in industrial scaled protein manufacture range from 2500 L to 6000 L of plasma per batch. In particular embodiments of the invention the precipitate is obtained from 2500 L to 6000 L of plasma. Some commercial manufacturing processes are capable of using even larger plasma donor pool sizes including up to 7500 L, up to 10000 L, and/or up to 15000 L of plasma.

The method and system of the invention can also be used not only for large industrial scale applications but as a stand-alone system and/or method for smaller production scale applications (where the starting material may be less than 200 L).

### Starting Material

The methods of the present invention advantageously enable the use of blood-derived plasma as the starting material for extraction of immunoglobulins and albumin. The plasma may be fresh plasma, “normal” plasma, “hyperimmune” plasma, cryo-poor plasma (also referred to as cryosupernatant), or cryo-rich plasma. Optionally, the plasma has been treated to remove components such as C1-inhibitor, PCC (Prothrombin Complex Concentrate) and/or AT-III. The plasma may be obtained from a number of donations and/or individuals, and pooled.

The term “cryosupernatant” (also called cryo-poor plasma, cryoprecipitate-depleted plasma and similar) refers to plasma (derived from either whole blood donations or plasmapheresis) from which the cryoprecipitate has been removed. Cryoprecipitation is the first step in most plasma protein fractionation methods in use today, for the large-scale production of plasma protein therapeutics. The method generally involves pooling frozen plasma that is thawed under controlled conditions (e.g. at or below 6° C.) and the precipitate is then collected by either filtration or centrifugation. The supernatant fraction, known to those skilled in the art as a “cryosupernatant”, is generally retained for use. The resulting cryo-poor plasma has reduced levels of Factor VIII (FVIII), von Willebrand factor (VWF), Factor XIII (FXIII), fibronectin and fibrinogen. Cryosupernatant provides a common feedstock used to manufacture a range of therapeutic proteins, including alpha 1-antitrypsin (AAT), apolipoprotein A-I (APO), antithrombin III (ATIII), prothrombin complex comprising the coagulation factors (II, VII, IX and X), albumin (ALB) and immunoglobulins such as immunoglobulin G (IgG).

The term “cryo-rich plasma” refers to plasma (derived from either whole blood donations or plasmapheresis) that has been frozen and then thawed, but from which the cryoprecipitate has not been removed.

Where plasma has been frozen for transport from a collection location, the frozen plasma is thawed and then collected in a pooling tank before centrifugation. The cryoprecipitate is removed by continuous centrifugation. The cryo-depleted plasma may be pumped into a stainless-steel fractionation tank and sampled for in-process controls

The plasma, whether pooled from more than one or several hundred individuals, or whether obtained from a single individual, may be hyperimmune plasma. For example, the plasma may be obtained from the blood of individual(s) who have/has mounted an immune response to an infection, and have recovered (and are therefore otherwise healthy individuals).

### Dynamic Filter Element

In any aspect of the present invention, the dynamic filter element filtration unit adapted for separating the soluble immunoglobulin from the insoluble albumin, is a dynamic cross flow filter element. In a preferred embodiment the dynamic cross flow filter element is a rotational cross flow filter element. More preferably the rotational cross flow filter element comprises a filter disc. The filter discs are usually mounted on a shaft member. In an embodiment the rotational cross flow filter element comprises at least one filter disc and at least one shaft member.

According to a preferred embodiment of any aspect of the present invention, the filter disc membrane is a ceramic membrane. More preferably the ceramic membrane has a pore size in the range of greater than or equal to 5 nm to less than or equal to 2 μm. In particular embodiments, the ceramic membrane has a pore size of from about 0.2 μm to 2 μm. In particular embodiments, the ceramic filter membrane has an average pore size in the range of greater than or equal to 5 nm to less than or equal to 200 nm (0.2 μm). In particular embodiments, the ceramic filter membrane has an average pore size in the range of greater than or equal to 50 nm to less than or equal to 100 nm. Such filter discs are supplied by Kerafol and Flowserve.

It will be understood that a plurality of filter disc membranes can be included in the dynamic filter element filtration unit adapted for separating the soluble immunoglobulins from the insoluble albumin. As such, the present methods contemplate the use of one, two, three, four, five, six or more filter disc membranes for separating the soluble immunoglobulins from the insoluble albumin. The plurality of filter disc membranes may have pore sizes that are the same or different.

The filtration unit, in preferred embodiments, comprises a pressure vessel. The suspension from the first tank can be continuously fed into the pressure vessel via an inlet port. An even distribution of the suspension in the vessel can be achieved using a distribution manifold. Hence in particular embodiments the pressure vessel comprises a distribution manifold. In some embodiments the first filtration unit comprises a rotational cross flow filter element. Preferably the filter element contains more than one filter disc evenly spaced along at least one hollow central collection shaft. The filter discs can be arranged either horizontally or vertically. When in the horizontal orientation they are spaced along a vertically orientated hollow collection shaft. The collection shaft and discs are rotatable. The suspension in the pressure vessel can then penetrate the outer membrane of the rotating filter discs so as to pass through into a hollow central portion of the disc which is in turn channeled into the central collection shaft. Typically, the filtrate (e.g., comprising the partially purified immunoglobulin) can then be removed from the shaft portion of the first filtration unit via a flanged port. Whilst the retentate (comprising insoluble components) remaining in the pressure housing can be fed out of the vessel via an outlet port. Generally, the retentate is recirculated to the first tank to dilute the suspension. In this way, the retentate from the first filtration unit can be utilised to dilute the suspension in the first tank.

Dynamic cross flow filtration such as rotational filtration provides maximum filter efficiency. The cross flow effect (tangential flow cleaning of the filter surface) is generated by rotating the filter discs and not by pumping large volumes across a fixed membrane as used in conventional (static) cross flow filtration systems. The extreme cross flow velocities generated at the surfaces of the rotating filter discs ensure a highly efficient cleaning of the filter surface, whilst consuming very low amounts of energy compared to conventional cross flow techniques.

Dynamic filter elements can also be employed to perform the continuous concentration process. Such dynamic filter elements will typically comprise one or more ultrafiltration or diafiltration membranes.

The cross flow filter element for performing the continuous concentration process may include a dynamic ultrafiltration filter device. Alternatively, the process comprises a static ultrafiltration device, for example Tangential Flow Filtration (TFF).

In preferred embodiments of the invention the dynamic cross flow filter element or the ultrafiltration filter device for performing the concentration process comprises a membrane with a molecular weight cutoff less than the molecular weight of the protein of interest (e.g., immunoglobulin G in the case of step e) of the third aspect of the invention). The molecule cutoff may be at least 3-fold lower than the molecular weight of the protein of interest (e.g. for a protein with a molecular weight of about 150 kDa the membrane may have a cut off of about 30-50 kDa). In these embodiments the membrane cutoff is selected to retain the protein of interest during the concentration process. As a general guide a nominal membrane cutoff at least 3-fold lower than the molecular weight of the protein of interest can be selected to ensure the protein is retained in the retentate.

In one alternative embodiment the dynamic cross flow filter element or the static ultrafiltration filter element for performing the concentration process comprises a membrane with a molecular weight cutoff greater than the molecular weight of the protein of interest. In such embodiments the nominal membrane cutoff is selected to ensure the protein of interest passes across the membrane and is collected in the filtrate rather than the retentate.

In embodiments where the cross flow filter element is dynamic, preferably the element is a rotational cross flow filter element, adapted for performing a continuous concentration process.

According to a further preferred embodiment, the filtration element for performing the continuous concentration process comprises a filtration membrane having an average pore size of between 5 nm to 5000 nm, preferably between 5 nm to 2000 nm, between 5 nm to 1000 nm, between 5 nm to 500 nm, between 5 nm to 200 nm, between 7 nm to 1000 nm, more preferably between 7 nm to 500 nm, even more preferably between 7 nm to 100 nm, most preferably between 7 nm to 80 nm. Of course, the average pore size can be in other combinations of the range given above. Filter manufacturers often assign terms like nominal or mean pore size ratings to commercial filters, which usually indicate meeting certain retention criteria for particles or microorganisms rather than the geometrical size of the actual pores.

In an embodiment, the filtration element performing the continuous concentration process is Dynamic Flow filtration or Tangential Flow Filtration (TFF) using a TFF membrane.

In a particular embodiment the rotational cross flow filter element for performing the continuous concentration process comprises a filter disc (such as a ceramic disc). In some embodiments the filter disc comprises a membrane with an average pore size of a microfilter. In other embodiments the filter disc comprises a membrane with an average pore size of an ultrafilter. In further embodiments, the filter disc comprises a membrane with an average pore size of a diafilter. In an embodiment the average pore size of the filter disc membrane is in a range from greater than or equal to 5 nm to less than or equal to 2 μm. In particular embodiments the average pore size of the filter disc membrane is in a range from greater than or equal to 50 nm to less than or equal to 500 nm (ie 0.5 μm). In some embodiments the filter disc membrane has an average pore size in the range of greater than or equal to 50 nm to less than or equal to 100 nm, or in the range of greater than or equal to 60 nm to less than or equal to 90 nm, or in the range of greater than or equal to 60 nm to less than or equal to 80 nm. In some embodiments the filter disc membrane has an average pore size of 60 nm or 80 nm.

In particularly preferred embodiments, the rotational cross flow filter element for performing the continuous concentration process comprises a plurality of ceramic discs with a pore size suitable for ultrafiltration and/or diafiltration. For example, the element preferably comprises at least one ceramic membrane having a pore size of 3 nm. Alternatively, the element preferably comprises at least one ceramic membrane having a pore size of 5 nm. Alternatively, the element preferably comprises at least one ceramic membrane having a pore size of 7 nm. Alternatively, the element preferably comprises at least one ceramic membrane having a pore size of 30 nm. The element may comprise a plurality of ceramic discs with varying pore sizes, including wherein the pore sizes are 3 nm and 5 nm. The element may comprise a plurality of ceramic discs with varying pore sizes, including wherein the pore sizes are 5 nm and 7 nm. The element may comprise a plurality of ceramic discs with varying pore sizes, including wherein the pore sizes are 3 nm and 30 nm. The element may comprise a plurality of ceramic discs with varying pore sizes, including wherein the pore sizes are 3 nm, 5 nm, 7 nm and 30 nm.

According to yet a further preferred embodiment, a filtration element for performing the continuous concentration process comprises an ultrafiltration device comprising a membrane in the form of a polymer membrane, such as polyethersulfone or regenerated cellulose.

Dynamic cross flow filtration such as rotational filtration provides maximum filter efficiency. The cross flow effect (tangential flow cleaning of the filter surface) is generated by rotating the filter discs and not by pumping large volumes across a fixed membrane as used in conventional (static) cross flow filtration systems. The extreme cross flow velocities generated at the surfaces of the rotating filter discs ensure a highly efficient cleaning of the filter surface, whilst consuming very low amounts of energy compared to conventional cross flow techniques.

In the dynamic cross flow filtration units and systems of the invention, the rotating ceramic filter discs are typically assembled in a pressurised housing. The design of the discs shows drainage channels in the inside. The filtrate is transported from the outside to the inside of the discs. The rotation of the discs generates shear forces on the membrane surface. With this technique an increase of a filter cake is avoided resulting in a high filtration flux. Some of main parameters of the rotation filtration is the rotation speed for rotating the ceramic filter disc, solid content (concentration of liquids due to the removal of filtrate) and transmembrane pressure. The transmembrane pressure is typically from 0.1 to 2.5 bar, preferably from 0.2 to 2.4 bar, more preferably from 0.4 to 2.0 bar, from 0.5 to 1.8 bar, from 0.6 to 1.6 bar, from 0.6 to 1.5 bar, from 0.7 to 1.5 bar, most preferably from 0.8 to 1.5 bar. According to another embodiment, a pressure of up to 2 bar, preferably between 0.1 to 2.0 bar, or about 1.5 bar, 1.0 bar or 0.5 bar is provided to filter units.

The temperature has an effect on the viscosity of a protein solution and therefore also has an effect on the flux upon filtration with a membrane. The starting suspension to be used in the method of the invention will preferably have a temperature within the range from 0° C. up to the temperature at which the protein concerned is denatured. The temperature is typically within the range of from about 18° C. up to about 40° C. In particular embodiments the temperature is within the range of from about 18° C. up to about 35° C. According to one preferred embodiment, the temperature of the suspension tank (i.e., the tank comprising the fatty acid, preferably caprylic (octanoic) acid for mixing with the plasma sample) is at a temperature of between about 18° C. to about 40° C. More preferably, the fatty acid, preferably caprylic (octanoic) acid is mixed with the plasma in the second suspension tank at a temperature of between about 18° C. to about 24° C., optionally about 21° C., about 22° C. or about 23° C.

According to a further preferred embodiment, the temperature in the filter units is controlled, preferably between 2° C. and 25° C., more preferably at about 18° C. to about 24° C. Such a temperature ensures an optimum extraction process and separation process while maintaining the bio-reactivity of the protein of interest throughout the processes.

Filtration is performed with a transmembrane filtration pressure that is the same as or below the level at which the membrane can withstand, depending on the material of the membrane to be used herein, for example with pressures of about 0.2 to about 3 bar. The transmembrane pressure is typically from 0.1 to 2.5 bar, preferably from 0.2 to 2.4 bar, more preferably from 0.4 to 2.0 bar, from 0.5 to 1.8 bar, from 0.6 to 1.6 bar, from 0.6 to 1.5 bar, from 0.7 to 1.5 bar, most preferably from 0.8 to 1.5 bar. According to another embodiment, a pressure of up to 2 bar, preferably between 0.1 to 2.0 bar, or about 1.5 bar, 1.0 bar or 0.5 bar is provided to filter units.

According to another embodiment, the continuous extraction process in the filter units adapted to separate impurities/precipitant from the first and second suspensions, is further assisted by regulating the flow rate and/or the residence time of the suspension or the solution into the filter units and/or the flow rate of the retentate/raffinate comprising impurities/precipitant and/or the flow rate of the first permeate/filtrate enriched for the protein of interest. For instance, in one embodiment, the linear velocity of the suspension or the solution into the pressure vessel (filtration process unit) can be about 0.27 to 1.66 m/s. In another example, the linear velocity of the retentate comprising impurities/precipitant can be 0.25 to 1.33 m/s. In another example, the linear velocity of the permeate/filtrate enriched for the protein of interest can be 0.03 to 0.33 m/s. Linear velocity multiplied by the cross-sectional area gives the volumetric flow rate. In addition, a turbulence can be created in the first process unit as a result of the speed of the rotating filter discs, wherein the speed (sometimes referred to as tangential speed) can be between about 1 to 7 m/s. According to an embodiment of the present invention, the speed of the rotating disc filters is between 1 to 10 m/s. In a preferred embodiment of the present invention, the speed of the rotating disc filters is between 5 to 7 m/s. More preferably the speed of the rotating disc filters is 7 m/s at 60 Hertz (800 rpm). The rotating speed of the rotational cross-filter element is between about 600 rpm (50 Hz) and about 1600 rpm (100 Hz), preferably between about 800 rpm (60 Hz) and about 1200 rpm (80 Hz), preferably about 800 rpm (60 Hz), about 1000 rpm (70 Hz) or about 1200 rpm (80 Hz). As used herein, the rotating speed in Hz is intended to refer to the speed of the motor. This can be correlated with the speed in rpm using an appropriate calibration curve.

This method allows a continuous extraction and a separation process to be realised for maximising the recovery of the protein of interest from the starting precipitate/material (i.e., the blood-derived plasma). Thanks to the extraction process, almost all the protein of interest is extracted and is recovered in subsequent stages. This method also allows the liquid or diluent e.g. buffer or water to be re-circulated in a closed system and hence the quantity of the liquid is maintained throughout the process while footprints (i.e. large tank volume) can be reduced.

In still further embodiments, the present invention includes a step of backflushing in conjunction with dynamic crossflow filtration, in order to flush contaminants that may have built up in the system. Typically, the methods and systems of the invention include alternating filtration and backflushing such that the filtration is temporarily paused (i.e., the feed pump is stopped) while a period of backflushing occurs, at regular intervals. where the flow of liquid into the filtration system is reversed,

It will be understood that the frequency, duration and flow rate of the backflushing can be adjusted in order to maximize filtration efficiency and the period of filtration before backflushing is required.

In certain preferred embodiments, the frequency of backflushing (and consequently, the period of filtration) is determined based on the total protein concentration or the number of impurities in the starting material. Consequently, it will be appreciated that backflushing will be required more frequently when filtering the suspension comprising plasma and fatty acid compared to the subsequent steps of further purifying the solubilised albumin preparation (which has relatively fewer impurities) is being filtered. In other words, as the total protein concentration and turbidity of the filtrate decreases, the frequency of the backflushing interval will also decrease (i.e. the period of time between backflushes increases and filtration can proceed for a longer period of time before a backflush is required).

Protein concentration and turbidity of the filtrate can be monitored by various methods known in the art. In certain embodiments, the methods of the invention and systems of the invention include the use of an in-line detection unit that enables measurement of protein concentration and/or turbidity as the filtrate passes into and/or out of the filtration unit. In further embodiments, a dual wavelength photometer can be employed to facilitate simultaneous assessment of protein concentration (e.g., by detecting absorbance of the solution at a wavelength suitable to detecting protein concentration, such as in the range of 260-280 nm, preferably about 280 nm) and solution turbidity (e.g., by detecting absorbance of the solution at a wavelength suitable for detecting light scattering caused by the presence of particulate matter, such as absorbance at a wavelength in the range of 400 nm to 900 nm, preferably about 600 nm to about 880 nm). Dual wavelength photometric devices for use in conjunction with chromatographic and filtration units are well known in the art.

In certain examples, the frequency of backflushing is at 15 second intervals, 30 second, 45 second, 60 second 75 second, 90 second, 105 second, 120 second, 135 second, 150 second, 200 second, 230 second, 260 second, 300 second, 330 second, 360 second, 400 second, 1000 second, 2000 second, 3000 second, 4000 second or greater intervals.

It will be appreciated that the duration of the backflushing interval will vary with the filtration area and number of discs requiring backflushing, The larger the filtration area, typically, the larger the volume of backflushing buffer that is required and there for the duration of the backflushing will also be dictated by the flow rate during backflushing. The skilled person will be well able to determine suitable duration, frequency, and flow rates for backflushing, depending on the size of the system and number of discs being employed. In certain examples, the duration of backflushing is approximately 5 seconds, approximately 10 seconds, approximately 15 seconds, approximately 30 seconds, approximately 45 seconds, approximately 60 seconds, or longer.

It will be appreciated that for practical reasons (and to maximize filtration efficiency) the duration of the backflushing interval is typically less than the duration of the filtration interval. In certain embodiments, the duration of the backflushing interval is at least one quarter, one eighth, one tenth, one sixteenth or smaller than the duration of the filtration interval.

It will further be appreciated that the flow rate that is used during backflushing may be the same or different to the flow rate used for filtration. In certain embodiments, the flow rate during dynamic filtration is in the range of approximately 15 to 100 L/hour, preferably in the range of about 20 to 50 L/hour (about 200 ml/min to about 1 L/min, preferably about 300 to about 900 ml/min, more preferably about 300 to 600 ml/min). Preferably the flow rate of backflushing is lower than the flow rate used for filtration such that in certain embodiments, the flow rate of backflushing is in the range of about 100 to about 400 times slower than the flow rate used for filtration.

In certain embodiment, the backflushing is performed with the same buffer comprised in the first or second suspensions. Alternatively, the backflushing may be performed using the permeate that is obtained during the concentration process (e.g., when using ultrafilitration coupled to dynamic cross flow filtration to concentrate the filtrate obtained from dynamic cross flow filtration).

### Protein Recovery and Further Processing

The concentration of protein(s) in a sample (e.g., in the supernatant or a subsequently purified preparation thereof) can be measured by any means known to persons skilled in the art. Examples of suitable assays include high pressure liquid chromatography (HPLC; e.g., size exclusion HPLC), enzyme-linked immunosorbent assay (ELISA), nephelometry and immunonephelometry. Such techniques can be used to assess purity of a sample (for example, to identify the presence of undesirable protein contaminants including proteases). In addition, gel electrophoresis like SDS-PAGE with staining and densitometry may be used to assess purity of the sample and detect the presence of contaminating proteins. A reducing agent such as dithiothreitol can be used with SDS-PAGE to cleave any disulfide-linked polymers.

In any embodiment, the temperature at which conductivity of a solution is measured can be between about 4° C. and about 37° C., preferably, wherein the temperature is between about 18° C. and about 25° C., or about 20° C. and about 25° C. (room temperature).

The ultrafiltered product comprising immunoglobulin (i.e., following continuous extraction and subsequent concentration), can later be subjected to further processing such as chromatography steps, virus inactivation steps, concentration and formulation so that the end product can be administered for example to the human body. The end product can be used in the treatment of immune conditions, particular autoimmune diseases and certain neurological diseases. These conditions include Rheumatoid arthritis, Systemic Lupus Erythematosus (SLE), Antiphospholipid syndrome, immune thrombocytopenia (ITP), Kawasaki disease, Guillain Barre syndrome (GBS), multiple sclerosis (MS), chronic inflammatory demyelinating polyneuropathy (CIDP), multifocal motor neuropathy (MMN), myasthenia gravis (MG), skin blistering diseases, scleroderma, Dermatomyositis, Polymyositis, Alzheimer's Disease, Parkinson's Disease, Alzheimer's Disease related to Downs Syndrome, cerebral amyloid angiopathy, Dementia with Lewy bodies, Fronto-temporal lobar degeneration or vascular dementia. In addition, the end IVIg and SCIg products can be used in other medical procedures such as in cell and organ transplant.

### EXAMPLES

### Example 1: Isolation of Purified IgG

Four independent experiments we conducted, in which IgG was purified from plasma according to the methods of the present invention.

In all 4 experiments, fresh plasma was diluted 1:2 in phosphate-acetate buffer (1 part plasma to 2 parts buffer), wherein the pH of the diluted plasma was approximately 4.6 to about 5.0 with a conductivity of 8-12 mS/cm. The diluted plasma was transferred to a tank (the suspension tank).

Octanoic acid was added to the diluted plasma in the suspension tank over a 20-60 minute period to the diluted buffer at an amount of at least 0.35 g/g total protein, with vigorous mixing to produce a plasma/octanoic acid emulsion. The emulsion was further incubated for 60-240 minutes at approximately 22° C. Without wishing to be bound by theory, the temperature, slow addition and vigorous mixing is believed to contribute to the thorough distribution of the octanoic acid through the diluted plasma. This, in turn, is believed to lead to more effective mixing of the octanoic acid with the albumin, and therefore more albumin contacting the octanoic acid, leading to a higher yield of precipitate. The end result is a more effective separation of the soluble immunoglobulins from the insoluble albumin-octanoic acid complex.

The resulting suspension was then fed into a continuous extraction filter unit, from which the retentate was recirculated back into the suspension tank, and from which the filtrate (comprising immunoglobulins) was fed into a second tank. The filtrate collected in the second tank was then fed into a second unit comprising the ultrafiltration and diafiltration system. The retentate of the UF/DF system flowed back into the second tank while the filtrate flowed back into the first tank.

The solution was diluted to a protein concentration of 20 g/L and the pH was adjusted to approximately pH 4.0 in the presence of polysorbate 80 (P80). The solution was subjected to further clarifying depth filtration. The resulting filtrate (referred to herein as the “clarified filtrate”) was further assessed to determine various product attributes, as described further below.

TABLE 1
Total yield and recovery of the IgG following UF/DF concentration
and following depth filtration each of the 4 experiments

| | IgG | | | | | | | |
|---|---|---|---|---|---|---|---|---|
| | Experiment 1 | | Experiment 2 | | Experiment 3 | | Experiment 4 | |
| | Yield | Recovery | Yield | Recovery | Yield | Recovery | Yield | Recovery |
| Step | (g/L) | (%) | (g/L) | (%) | (g/L) | (%) | (g/L) | (%) |
| Plasma Pool | 8.6 | 100 | 8.4 | 100 | 6.7 | 100 | 6.6 | 100 |
| UF/DF concentrate | 8.1 | 94 | 8.0 | 94.8 | 6.2 | 99.3 | 6.2 | 93.7 |
| Clarified filtrate | 8.0 | 93.6 | 8.2 | 97.9 | 6.2 | 92.2 | 6.2 | 94.4 |

These results demonstrate that the methods of the invention provide a highly efficient approach for the isolation of high levels of IgG directly from a sample of plasma. The yield of IgG is high, and the recovery of IgG is also consistent with other commercial manufacturing processes for the isolation of IgG. The immunoglobulin levels were measured by immunonephelometry.

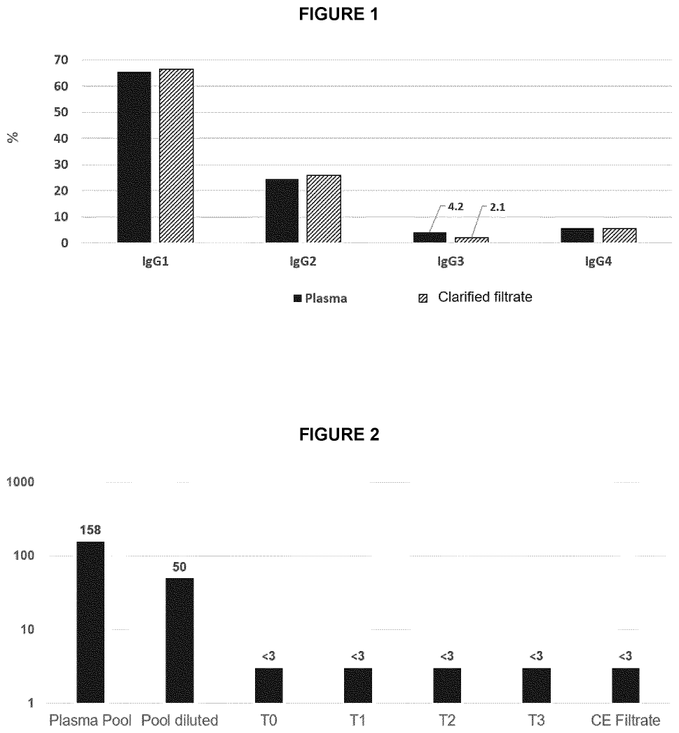

The distribution of the IgG subclasses was determined by immunonephelometry and the results, shown in FIG. 1, indicate that the distribution of IgG subclasses in the purified immunoglobulin preparation is similar to the distribution found in plasma.

### Example 2: Determination of Protease and Other Contamination in IgG Preparation

The concentration and degree of protease activity in the clarified filtrate was determined using standard methods. Briefly, a chromogenic substrate-based assay was used. Serine protease activity was measured by the ability of protein concentrate to cleave the chromogenic substrate Ile-Pro-Arg-pNA (S-2288). During this reaction p-Nitroanilin (pNA) is released which is measured in a photometer at 405 nm. Serine protease activity was measured in conditions where the pH was 8.4 at 37° C. Kallikrein-like activity was measured by cleavage of the chromogenic substrate H-D-Pro-Phe-Arg-pNA (S-2302). During this reaction p-Nitroanilin (pNA) is released which is measured in a kinetic mode by a photometer at 405 nm.

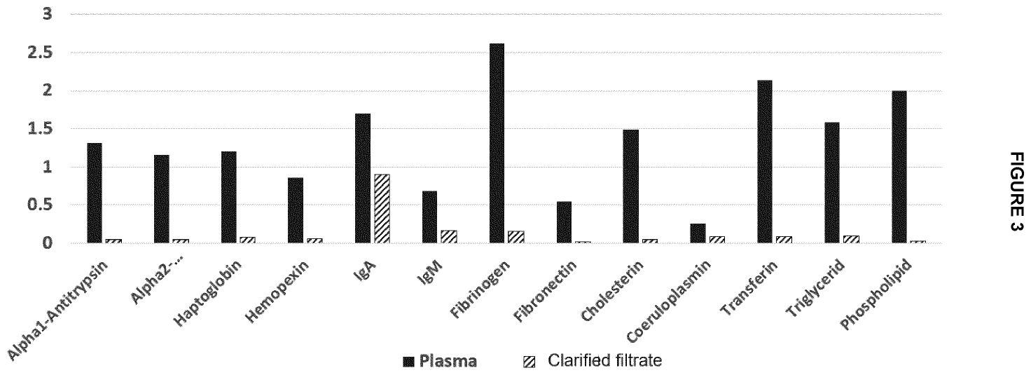

As shown in FIG. 2, the methods of the present invention effectively eliminate protease contamination.

Other impurities, such as IgA, IgM, alpha1-antitrypsin were determined using standard techniques. Impurities such as IgA, IgM and ceruloplasmin were found to be present at lower but still detectable levels. Other impurities were determined to be below detectable limits (α-1-antitrypsin, α-2-macroglobulin, haptoglobin, hemopexin, fibrinogen, fibronectin, cholestrin, transferrin, triglyceride and phospholipid). The results are shown in FIG. 3. The protein impurities were determined by immunonephelometry, while the cholestrin triglyceride and phospholipid levels were determined using an enzymatic assay.

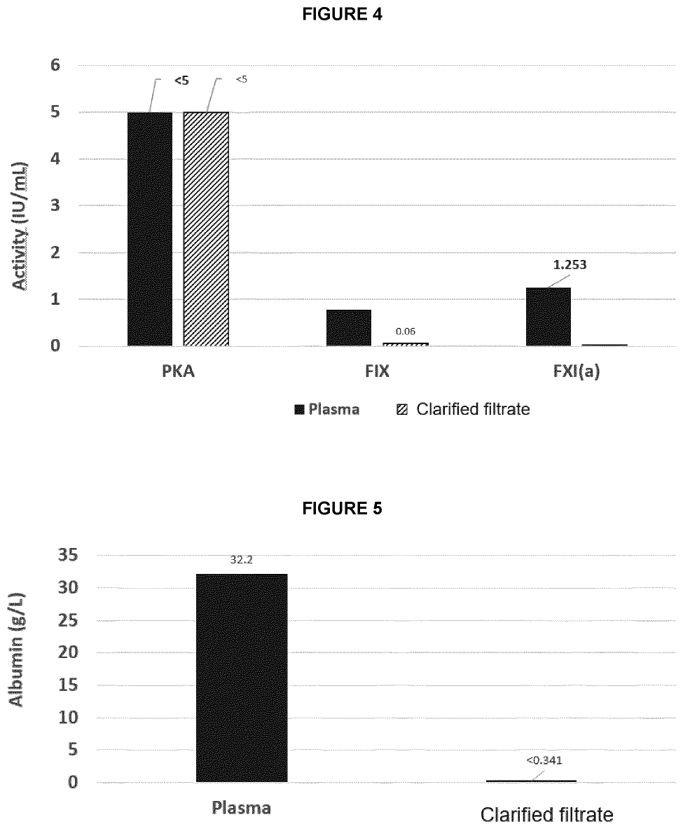

The amounts of pre-kallikrein activator (PKA), Factor IX and Factor XI (a) were also determined. These results are shown in FIG. 4. PKA was measured by a chromogenic substrate (as discussed above), FIX was measured by ELISA and FXI (a) was measured using an activated Partial Thromboplastin Time (aPTT) assay.

The amount of albumin (g/L) in the starting plasma material was compared to the amount of albumin present in the IgG preparation described in Example 1 (i.e., the clarified filtrate). Total albumin present in plasma was approximately 32.2 g/L. The amount of albumin in the IgG preparation of Example 1 was less than 0.341 g/L, consistent with typical amounts of albumin found in commercial grade preparations of IgG. Results are shown in FIG. 5.

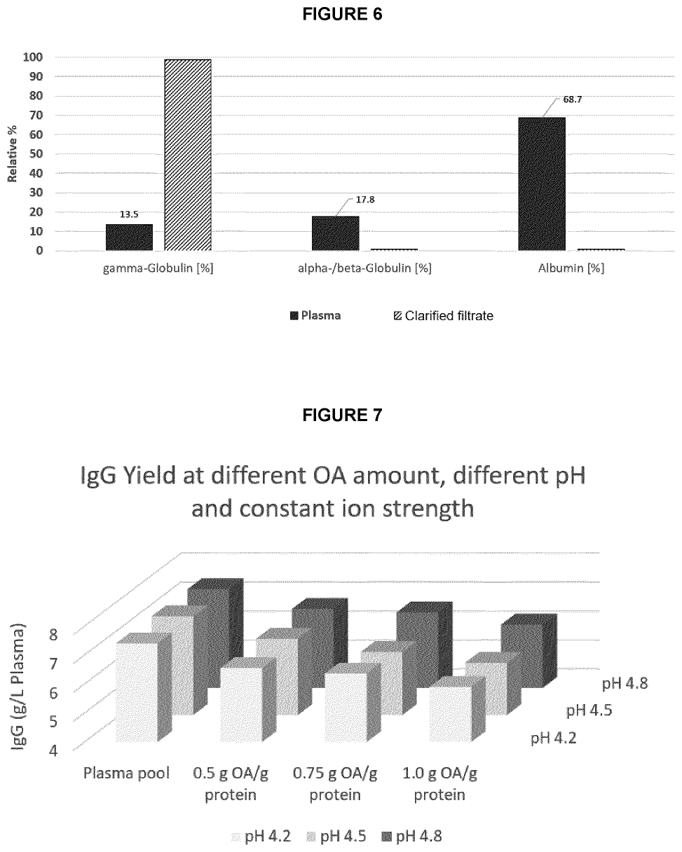

Further results shown in FIG. 6 indicate that the immunoglobulin preparation generated in Example 1 is highly enriched for gammaglobulins with only low levels of contamination alpha/beta-globulins and albumin. The gammaglobulin and alpha-/beta-globulin levels were determined using cellulose acetate/agarose electrophoresis.

### Example 3: Isolation of Purified Albumin

Following the completion of Example 1, the residual suspension in the first tank, was mixed with phosphate buffer (pH 7.1 to 7.4), and the pH adjusted to between 6.4 to 6.7. The resulting solution comprised soluble albumin. Alternatively, following completion of Example 1, the residual suspension in the first tank had a pH adjustment to 6.4 to 7.2 (preferably 6.8 to 7.2) with 1M sodium hydroxide. Also contemplated is a combination of sodium hydroxide and phosphate buffer at 0.12M.

The solution was further processed to achieve a total protein concentration of about 0.2 g/L to below 1.0 g/L (or about 0.2 g/L to about 0.5 g/L).

Briefly, the solution of albumin was fed into a continuous extraction filter unit, from which the retentate was recirculated back into the tank, and from which the filtrate (comprising albumin) was fed into a second tank. The filtrate collected in the second tank was then fed into a second unit comprising a ultrafiltration and diafiltration system. The retentate of the UF/DF system flowed back into the second tank while the filtrate flowed back into the first tank.

The retentate of the UF/DF, comprising concentrated albumin was completed once the desired protein concentration was reached. This sample is referred to in the table below as “Crude albumin after CE”.

TABLE 2
Total concentration, amount and recovery of the albumin
was determined (average of 2 experiments shown)

| | | Total | Protein | Albumin | Albumin |
|---|---|---|---|---|---|
| | Volume | protein conc. | amount | conc. | amount |
| Step | (L) | (g/L) | (g) | (g/L) | (g) |
| Plasma | 2.9 | 57.7 | 161.2 | 31.9 | 92.7 |
| Crude albumin after CE | 4.5 | 19.6 | 88.6 | 18.1 | 83.9 |
| Crude Albumin Recovery % | | | | | 90.2% |

### Example 4: Evaluation of Parameters

A number of independent experiments were conducted, to determine the optimum parameters within which in IgG or albumin is purified from plasma according to the methods of the present invention.

In all experiments, fresh plasma was diluted 1:3 in phosphate-acetate buffer (1 part plasma to 2 parts buffer), wherein the pH of the diluted plasma was approximately 4.6 to about 5.0 with a conductivity of 8-12 mS/cm.

Lab scale experiments were performed to assess the effect of parameters such as ionic strength, pH, dilution ratio diluent type (and/or diluted acetic acid on impurity removal, IgG and Albumin recovery at different OA concentrations.

### Yield and Impurities at Constant Ionic Strength and Various pH and OA Amounts

One kilogram of pooled plasma was diluted with 100 mM sodium acetate, pH 4.0 (ratio: 1:3). The diluted plasma was divided into 3 equal parts and the pH adjusted to the desired pH (4.2; 4.5 and 4.8) by the addition of concentrated acetic acid dropwise with thorough mixing. Total protein concentration was determined by measuring the absorption at A280. The conductivity of each aliquot was adjusted to within the range of 8.5+/−10 mS/cm using Acetate buffer

Octanoic acid was added to the diluted plasma over a 20-40 minute period to the diluted buffer at an amount of at least 0.35 g/g total protein, with vigorous mixing to produce a plasma/octanoic acid emulsion, with a final concentration of 0.5, 0.75 or 1.0 g/g total protein, respectively. The emulsion was further stirred for 60-180 minutes and then incubated with Celpure 100 for 15 minutes at 5 g/kg of solution. Filtration was then performed using CH9 filter layers.

The subsequent washing was performed using dilution buffer, previously adjusted to the same pH and conductivity under which the experiments were carried out, at 20% of the starting volume.

The clarified protein solution was then Ultra-Diafiltred up to 15-20 g/L. The pH of the solution was adjusted during the diafiltration to 4.00±0.20. The protein solution is then incubated at 37° C. for 9±1 h, following which the pH is shifted to 5.80±0.10. Following a subsequent deep filtration step the solution is loaded onto a strong anion exchange column, and the purified IgG is collected in flow through and the pH adjusted to 4.80±0.10

TABLE 3
Experimental conditions tested with constant conductivity:

| Constant | | | |
|---|---|---|---|
| conductivity | | | |
| (8.5 ± 1 mS/cm) | OA Amount (g OA/g protein) | | |
| pH 4.20 | 0.5 | 0.75 | 1.0 |
| pH 4.50 | 0.5 | 0.75 | 1.0 |
| pH 4.80 | 0.5 | 0.75 | 1.0 |

TABLE 4
IgG yield at constant ionic strength and various
pH and OA concentrations (IgG (g/L plasma)).

| | pH 4.2 | pH 4.5 | pH 4.8 |
|---|---|---|---|
| Plasma pool | 7.4 | 7.4 | 7.4 |
| 0.5 g OA/g protein | 6.55 | 6.63 | 6.72 |
| 0.75 g OA/g protein | 6.36 | 6.17 | 6.6 |
| 1.0 g OA/g protein | 5.90 | 5.8 | 6.18 |

TABLE 5
Albumin precipitation at constant ionic strength
and various pH and OA concentrations

| Albumin (g/L plasma) | pH 4.2 | pH 4.5 | pH 4.8 |
|---|---|---|---|
| Plasma pool | 35.1 | 35.1 | 35.1 |
| 0.5 g OA/g protein | 0.14 | 0.59 | 2.92 |
| 0.75 g OA/g protein | 0.14 | 0.28 | 0.16 |
| 1.0 g OA/g protein | 0.14 | 0.2 | 0.16 |

### Results

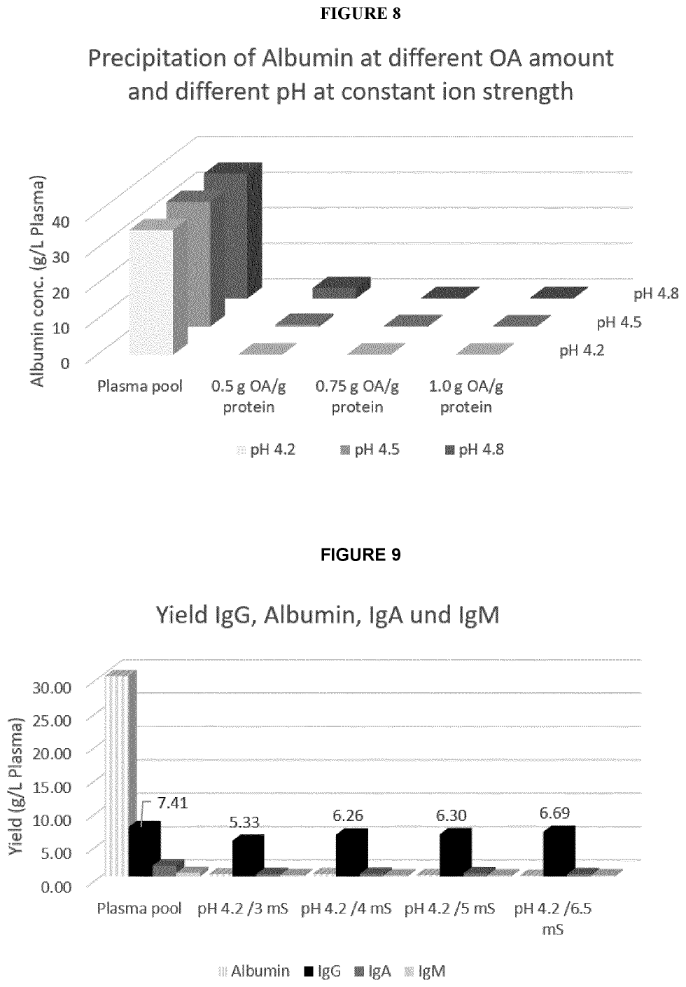

At low pH (4.2) and low concentrations of octanoic acid (0.5 g/g protein) almost all of the albumin is precipitated compared to a pH of 4.8 (FIG. 8). At pH 4.8 significant amounts of albumin is still present in the clarified and diafiltred protein solution post octanoic acid treatment. IgG yield is higher at pH 4.8 compared to the pH 4.2 (FIG. 7).

### Lower Conductivity and Higher Octanoic Acid

The effect of a lower conductivity (2 to 5 mS/cm) at pH 4.20 at high octanoic acid concentration (e.g. 1.0 g/g protein) was investigated.

The precipitating ability of octanoic acid at a concentration of 1 g/g protein) and a pH of 4.2 was investigated, at varying ionic strengths of 3; 4; 5 and 6.5 mS/cm.

TABLE 6
Results of IgG-, Albumin-, IgA und IgM Yield (g/L Plasma)

| Yield (g/L Plasma) | Albumin | IgG | IgA | IgM |
|---|---|---|---|---|
| Plasma pool | 33.10 | 7.41 | 1.65 | 0.515 |
| pH 4.2/3 mS | 0.33 | 5.33 | 0.347 | 0.189 |
| pH 4.2/4 mS | 0.34 | 6.26 | 0.375 | 0.150 |
| pH 4.2/5 mS | 0.19 | 6.30 | 0.450 | 0.145 |
| pH 4.2/6.5 mS | 0.11 | 6.69 | 0.345 | 0.162 |

### Results

The results (Table 6 and FIG. 9) clearly show that with low conductivity (≤5 mS/cm), the IgG yield is low compared to higher conductivity (≥6 mS/cm). Albumin content in the clarified concentrated octanoic acid filtrate is comparable to that at higher conductivity.

### Higher Octanoic Acid and Different pH

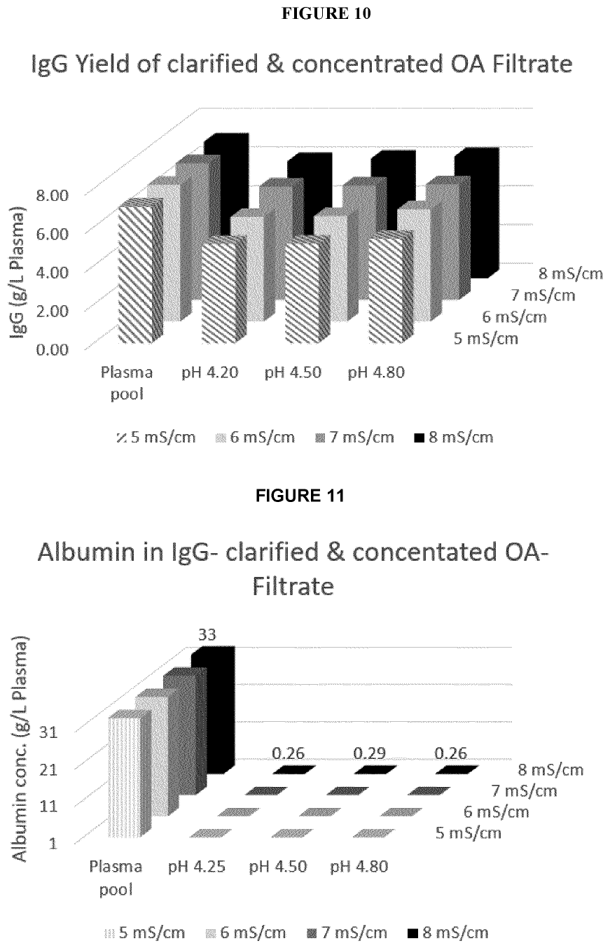

Octanoic acid at a concentration of 0.55 g/g protein was investigated at different pHs (4.2, 4.5, and 4.8) and different ionic strengths (5; 6; 7 and 8 mS/cm)—see Tables 7 to 10 below, and FIGS. 10 to 13.

TABLE 7
IgG yield

| | Conductivity (mS/cm) | | | |
|---|---|---|---|---|
| IgG (g/L plasma) | 5 mS/cm | 6 mS/cm | 7 mS/cm | 8 mS/cm |
| Plasma pool | 7.06 | 7.06 | 7.06 | 7.06 |
| pH 4.20 | 5.13 | 5.41 | 5.85 | 6.05 |
| pH 4.50 | 5.14 | 5.44 | 5.91 | 6.18 |
| pH 4.80 | 5.40 | 5.80 | 5.97 | 6.30 |

TABLE 8
Albumin in IgG-clarified and concentrated OA-filtrate

| Albumin (g/L plasma) | 5 mS/cm | 6 mS/cm | 7 mS/cm | 8 mS/cm |
|---|---|---|---|---|
| Plasma pool | 33 | 33 | 33 | 33 |
| pH 4.25 | 0.31 | 0.3 | 0.28 | 0.26 |
| pH 4.50 | 0.39 | 0.36 | 0.28 | 0.29 |
| pH 4.80 | 0.32 | 0.28 | 0.23 | 0.26 |

TABLE 9
IgA in IgG-clarified and concentrated OA-filtrate

| IgA (g/L plasma) | 5 mS/cm | 6 mS/cm | 7 mS/cm | 8 mS/cm |
|---|---|---|---|---|
| Plasma pool | 1.65 | 1.65 | 1.65 | 1.65 |
| pH 4.25 | 0.53 | 0.58 | 0.54 | 0.56 |
| pH 4.50 | 0.66 | 0.60 | 0.69 | 0.71 |
| pH 4.80 | 0.66 | 0.58 | 0.68 | 0.74 |

TABLE 10
IgM in IgG-clarified and concentrated OA-filtrate

| IgM (g/L plasma) | 5 mS/cm | 6 mS/cm | 7 mS/cm | 8 mS/cm |
|---|---|---|---|---|
| Plasma pool | 0.628 | 0.628 | 0.628 | 0.628 |
| pH 4.25 | 0.28 | 0.33 | 0.36 | 0.33 |
| pH 4.50 | 0.28 | 0.29 | 0.29 | 0.33 |
| pH 4.80 | 0.19 | 0.17 | 0.17 | 0.26 |

### Results

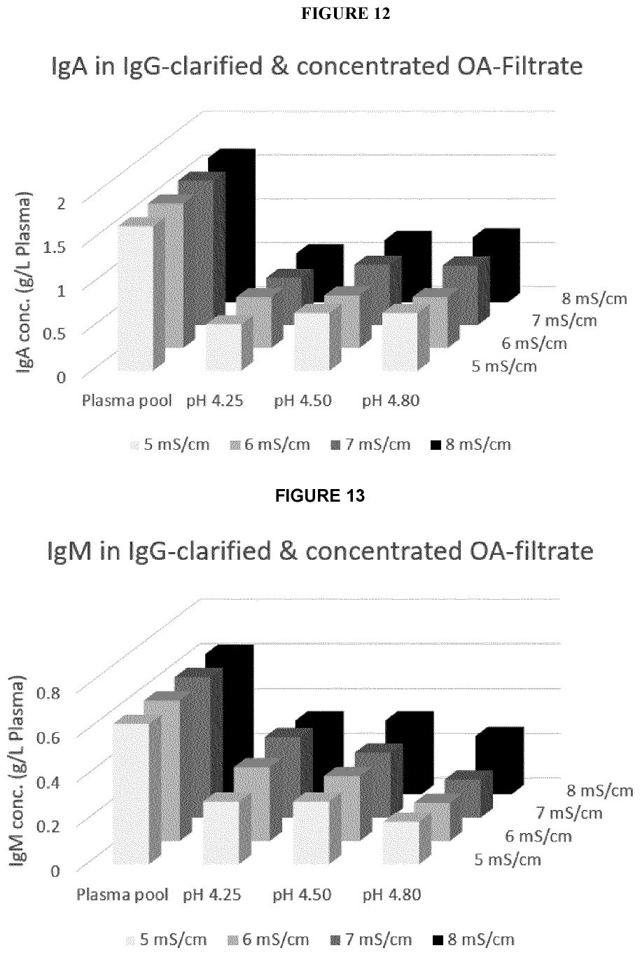

The data show that the IgG yield at pH 4.8 is higher at pH's 4.2 and 4.5, regardless of octanoic acid concentration (Table 7 and FIG. 10). The residual amount of albumin (Table 8 and FIG. 11), IgA (Table 9 and FIG. 12) and IgM (Table 10 and FIG. 13) in the clarified and concentrated IgG solution is relatively low and can be easily eliminated in further process steps (e.g. main chromatography purification) downstream of this octanoic acid step. Thus, IgG solution can be further purified in current process sequentially.

At pH 4.2, especially for the precipitation of albumin, at higher conductivity, the IgG yield is still marginally lower than at pH 4.8, but still very acceptable. It is also evident from the data that at a suitable octanoic acid concentration (e.g. range 0.50-0.55 g/g protein), it is possible appreciable amounts of albumin are precipitated by octanoic acid, so that albumin can be recovered too at high yield and purity

### Example 5: Continuous Filtration

In this experiment, the starting Plasma Pool was diluted in acetate, or phosphate/acetate Buffers with varying ionic strengths (60 mM, 80 mM or 100 mM) and pH values (4.0, 4.2, 4.5, 4.8 or 5.0).

One part of plasma was diluted with two parts of buffer, using an impeller mixer.

The diluted plasma was transferred to a tank (the suspension tank). Octanoic acid was added to the diluted plasma in the suspension tank over a 20-40 minute period to the diluted plasma at an amount of 0.50 g OA/g protein, 0.75 OA/g protein and 1.00 g OA/g protein, with vigorous mixing to produce a plasma/octanoic acid emulsion.

The resulting suspension was then fed into a continuous extraction filter unit, from which the retentate was recirculated back into the suspension tank, and from which the filtrate (comprising immunoglobulins) was fed into a second tank. The filtrate collected in the second tank was then fed into a second unit comprising the ultrafiltration and diafiltration system. The retentate of the UF/DF system flowed back into the second tank while the filtrate flowed back into the first tank.

The transmembrane pressure (TMP) is regulated to ensure that the permeate (filtrate) flow from the second unit, equals the filtrate flow from the first unit, ensuring a constant volume in the first tank during the extraction process.

The filtration unit is stopped once the protein concentration in the filtrate is below a defined threshold whereby the final dilution ratio of ≥1:X is reached.

The dilution ratio (1:X) can be variable, depending on the initial protein concentration of the plasma pool, the OA amount used as well as the desired protein concentration threshold of the residual OA-suspension to be reached. Almost the average final dilution ratio is ≥1:20. In certain embodiments the final dilution ratio was ≥1:12 and ≥1:15, or more (e.g. 1: ≥30).

The concentration step is completed (i.e., the UF concentrate) once the protein concentration reached about 25 to about 30 g/L. During this final concentration, the permeate flows to waste.

Then diafiltration is started. The concentrated protein solution is then diafiltered against 10× volume with WFI, during the diafiltration the pH is slowly lowered using 0.2M hydrochloric acid (HCl) such that a pH of 4.0±0.2 is adjusted towards the end of the diafiltration.

The diafiltered solution was diluted to a protein concentration of 20±2 g/L and the pH was adjusted to approximately pH 4.0±0.2. The solution was subjected to further clarifying depth filtration. The resulting filtrate (referred to herein as the “clarified filtrate”) was further assessed to determine various product attributes, as described further below in Example 6.

### Example 6: Continuous Filtration

2.6 L Cryo-rich plasma was diluted with 1.26 liters of 0.2 M Acetic acid to pH 4.8 and a conductivity of 7.5 mS/cm.

Octanoic acid (0.447 g/g protein) was added slowly with vigorous stirring, over a time period of 60 min to form an octanoic acid suspension with aa pH of 4.76 and a conductivity of 7.72. The octanoic acid suspension was incubated at 20° C. for 3.25 h before being transferred to the Feed Tank of the continuous extraction system of Example 1. The continuous extraction system was treated with 100 mM sodium acetate buffer (pH 4.8), refilled as well as the backflash tank.

The octanoic acid suspension was recirculated over a period of 15 minutes without filtration and then the filtration was started. Backflash time was 15 seconds and filtration time was 5 minutes. After about 5 minutes the TFF system (an exemplary system as shown in FIG. 16) was started and recirculation carried out for about 4 h, as described above.

Once the protein concentration in the feed tank reached 0.2-0.5 g/L the permeate from the TFF system was drained to waste.

The solution was diluted to a protein concentration of 20 g/L and the pH was adjusted to approximately pH 4.0 in the presence of polysorbate 80 (P80). The solution was subjected to further clarifying depth filtration.

50 mg DEAE A-50/g protein was added to the clarified filtrate solution, the pH adjusted to 5.8 using Tris base, and the solution stirring for 60 minutes, after which the protein solution was loaded on strong anion exchanger. The flow though was collected and the pH adjusted to 4.8 using 0.2M HCl. The resulting flow through was further assessed to determine various product attributes, as described further below.

TABLE 11
IgG yield and the other main impurities in the clarified
and concentrated solution, and post chromatography

| g/L plasma | IgG | IgA | IgM | Albumin |
|---|---|---|---|---|
| Plasma pool | 6.71 | 1.37 | 0.51 | 30.4 |
| Clarified solution | 5.98 | 0.63 | 0.28 | 0.9 |
| Post chromatography | 5.72 | 0.001 | <0.0002 | <0.339 |

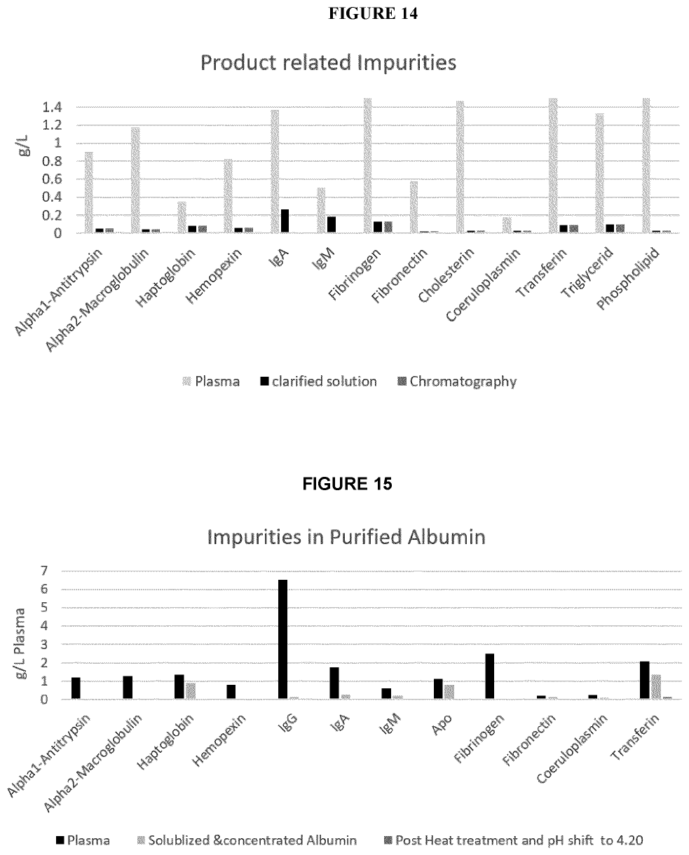

All other product related impurities are below the quantification limit (see FIG. 14).

### Example 7: Further Experiments Exploring Parameters

In a series of experiments (referred to below as examples 9 to 15), several parameters were investigated, which have an influence on the formation of an insoluble albumin-octanoic acid complex. These parameters are: pH, ionic strength, OA concentration, dilution factor, and the total recirculation volume (final dilution ratio) etc. These parameters lead to a more effective separation of the soluble immunoglobulins from the insoluble albumin-octanoic acid complex.

The following table (Table 12) contains the experimental test conditions

| | | Dilution | | | | Conductivity | OA |
|---|---|---|---|---|---|---|---|
| | Plasma | of | | Total | pH | (mS/cm) | amount |
| Example | pool | plasma | Buffer | Recirculation | OA | OA | (g/g |
| No | type | (1 + X) | used | volume (L) | suspension | Suspension | protein) |
| 9 | Cryo | 1 + 2 | 60 mM | 80 | 4.5 | 6.42 | 1.1 |
| | poor | | Acetate, | | | | |
| | | | (pH 4) | | | | |
| 10 | Cryo | 1 + 1.9 | 60 mM | 120 | 4.46 | 6.25 | 1.0 |
| | poor | | Acetate, | | | | |
| | | | (pH 4) | | | | |
| 11 | Cryo | 1 + 2.35 | 60 mM | 70 | 4.2 | 6.3 | 0.7 |
| | rich | | Acetate | | | | |
| | | | (pH 4) | | | | |
| 12 | Cryo | 1 + 2.93 | 60 mM | 70 | 4.25 | 6.3 | 0.6 |
| | rich | | Acetate | | | | |
| | | | (pH 4) | | | | |
| 13 | Cryo | 1 + 0.59 | 100 mM, | 60 | 4.80 | 6.9 | 0.7 |
| | poor | | Acetate | | | | |
| | | | (pH 4.8) | | | | |
| 14 | Cryo | 1 + 0.50 | 100 mM, | 86 | 4.7 | 7.0 | 1.0 |
| | rich | | Acetate | | | | |
| | | | (pH 4.8) | | | | |
| | | | & 0.2M | | | | |
| | | | Acetic | | | | |
| | | | acid | | | | |
| 15 | Cryo | 1 + 1.58 | 80 | 97 | 4.7 | 7.1 | 0.45 |
| | rich | | &100 | | | | |
| | | | mM, | | | | |
| | | | Acetate | | | | |
| | | | (pH | | | | |
| | | | 4.0/4.8) | | | | |

The results show very good agreement with the lab-scale experiments. Table 13 indicates the IgG yield and the major impurity and residual albumin content in the clarified and concentrated OA filtrate.

The data shows a consistent IgG yield with an average of 88.6% (range: 84.2-93.0%). The average residual albumin in Clarified solution is below 0.2 g albumin per liter plasma with the exception of Example 15. This is conceivable due to the higher IgM content in the plasma pool and the lower OA concentration

TABLE 13
IgG Yield and main impurities in Clarified OA Filtrate

| | | IgG | IgA | IgM | Albumin |
|---|---|---|---|---|---|
| Example | | (g/L | (g/L | (g/L | (g/L |
| No. | | Plasma) | Plasma) | Plasma) | Plasma) |
| 9 | Plasma pool | 6.51 | 1.76 | 0.61 | 35.8 |
| | Clarified solution | 5.62 | 0.65 | 0.20 | <0.11 |
| 10 | Plasma pool | 7.55 | 1.67 | 0.53 | 33.7 |
| | Clarified solution | 6.63 | 0.74 | 0.27 | <0.19 |
| 11 | Plasma pool | 7.29 | 2.03 | 0.71 | 33.2 |
| | Clarified solution | 6.25 | 0.63 | 0.29 | <0.19 |
| 12 | Plasma pool | 7.24 | 1.54 | 1.08 | 33.7 |
| | Clarified solution | 6.37 | 0.50 | 0.44 | <0.15 |
| 13 | Plasma pool | 6.53 | 1.64 | 0.63 | 30.0 |
| | Clarified solution | 5.53 | 0.62 | 0.25 | 0.20 |
| 14 | Plasma pool | 8.1 | 1.78 | 0.85 | 32.7 |
| | Clarified solution | 7.48 | 0.85 | 0.38 | <0.15 |
| 15 | Plasma pool | 5.58 | 1.24 | 1.2 | 33.8 |
| | Clarified solution | 5.20 | 0.78 | 0.69 | 2.57 |

After recovery of the soluble protein-containing component (immunoglobulin G and others (components such as IgA and IgM). Any residual suspension, and/or the first retentate, can be further processed to obtain purified albumin.

### Example 8: Further Processing of Albumin

The residual suspension, in the first retentate tank contains the insoluble Albumin-OA complex. The residual suspension contains IgG of less than 0.00035 g/L, IgA of less than 0.0002 g/L and IgM of less than 0.0002 g/L.

In order to disrupt binding between Albumin and the OA, the pH of the residual suspension was adjusted to 6.4-6.7, preferable 6.8-7.2 with 1M sodium hydroxide. After mixing and the pH of the solubilized Albumin is stable (30-60 min). The solution is feed in into the continuous extraction system to produce a retentate depleted of albumin and a filtrate enriched in albumin. The filtrate is continuously concentrated up to 20-45 g/L protein, using a TFF membrane (an exemplary system is shown in FIG. 16).

The concentrated albumin solution is then heated at temperature in the range of 60-65° C. for a time period over 90 min.

The pH of the solution was adjusted to 4.20 with 1 M Hydrochloric acid and the concentrated albumin solution is then cooled to 4° C. Precipitate is formed, which is dissolved during disruption of Albumin-OA complex at the mentioned pH.

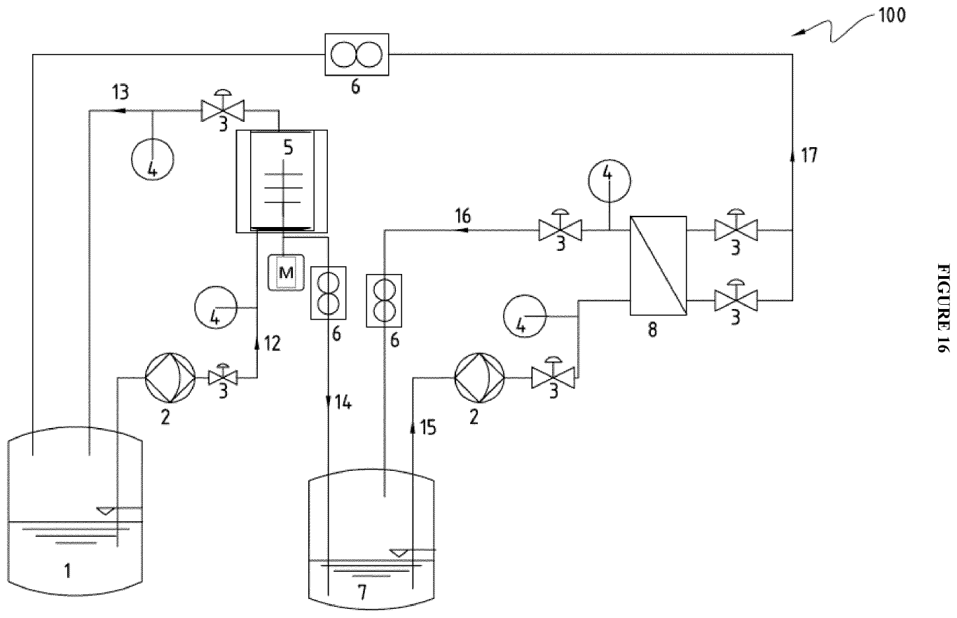

The precipitate was removed by filtration. The protein in the filtrate consisting mainly of Albumin (purity greater than 98% at Albumin yield of 90%). Table 14 and FIG. 15 show the impurity profile of heat treated albumin.

TABLE 14
impurities in heat treated Albumin

| | Alpha1- | Alpha2- | | | | |
|---|---|---|---|---|---|---|
| g/L plasma | Antitrypsin | Macroglobulin | Haptoglobin | Hemopexin | IgG | IgA |
| Plasma | 1.18 | 1.26 | 1.33 | 0.787 | 6.51 | 1.76 |
| Solublized & | 0.06 | 0.045 | 0.90 | <0.14 | 0.11 | 0.265 |
| concentrated | | | | | | |
| Albumin | | | | | | |
| Post Heat | 0.03 | <0.02 | <0.03 | <0.02 | 0.003 | 0.001 |
| treatment | | | | | | |
| and pH shift | | | | | | |
| to 4.20 | | | | | | |

| g/L plasma | IgM | Apo | Fibrinogen | Fibronectin | Coeruloplasmin | Transferin |
|---|---|---|---|---|---|---|
| Plasma | 0.61 | 1.12 | 2.49 | 0.191 | 0.233 | 2.08 |
| Solublized & | 0.181 | 0.804 | <0.05 | 0.11 | 0.07 | 1.35 |
| concentrated | | | | | | |
| Albumin | | | | | | |
| Post Heat | 0.0002 | <0.02 | <0.05 | <0.01 | 0.03 | 0.13 |
| treatment | | | | | | |
| and pH shift | | | | | | |
| to 4.20 | | | | | | |

It will be understood that the invention disclosed and defined in this specification extends to all alternative combinations of two or more of the individual features mentioned or evident from the text or drawings. All of these different combinations constitute various alternative aspects of the invention.

1-78. (canceled)

79. A method for purifying immunoglobulins from plasma, the method comprising:
(a) in a first tank, mixing a sample of blood-derived plasma comprising immunoglobulins and albumin with a medium chain fatty acid under conditions comprising a pH of about 4.6 to about 5.0 to permit selective precipitation of albumin from the sample, thereby forming a suspension comprising soluble immunoglobulins and insoluble albumin;
(b) feeding the suspension from the first tank into a first filtration unit comprising a dynamic filter element, thereby obtaining a first retentate comprising the insoluble albumin and a first filtrate comprising the soluble immunoglobulins; and
(c) recovering the first filtrate comprising soluble immunoglobulins in a second tank

80. The method of claim 79, wherein the blood-derived plasma is human blood-derived plasma and the immunoglobulins purified from the blood-derived plasma comprise human immunoglobulin G (IgG).

81. The method of claim 79, wherein the conditions in the first tank further comprise a conductivity of between about 8 mS/cm to about 12 mS/cm.

82. The method of claim 79, wherein the method further comprises streaming first retentate obtained at step (b) into the first tank, thereby diluting the suspension of step (a).

83. The method of claim 79, wherein the method further comprises concentrating the first filtrate via a second filtration unit comprising a dynamic filter element or Tangential Flow Filtration (TFF), thereby obtaining a second retentate enriched for immunoglobulins and a second filtrate depleted of immunoglobulins.

84. The method of claim 83, wherein the method further comprises streaming second filtrate depleted of immunoglobulins into the first tank, thereby diluting the suspension of step (a).

85. The method of claim 83, wherein the method further comprises streaming second retentate enriched for immunoglobulins into the second tank.

86. The method of claim 79, further comprising adjusting the pH of the first retentate to a pH of 6.4 to 7.2, thereby obtaining a solution comprising solubilized albumin.

87. The method according to claim 86, further comprising feeding the solution comprising solubilized albumin into a further filtration unit comprising a dynamic filter element, thereby obtaining a further retentate depleted of albumin and a further filtrate enriched in albumin.

88. The method according to claim 87, further comprising:
heating the further filtrate enriched in albumin at a temperature of 60° C. to 65° C. for a time period of 90 min or longer;
after the heating step, adjusting the pH of the further filtrate enriched in albumin to about 4.20 to precipitate any proteins denatured during the heating step, and removing the precipitate by filtration to obtain a filtrate comprising purified albumin.

89. The method according to claim 88, further comprising cooling the further filtrate enriched in albumin to 4° C. while the pH is adjusted.

90. The method of claim 88, wherein the filtrate comprising purified albumin has an albumin purity equal to or greater than 95%, 96%, 97%, or 98% total protein and an albumin yield equal to or greater than 85%, 86%, 87%, 88%, 89% or 90%.

91. The method of claim 79, wherein the dynamic filter element of the first filtration unit is a dynamic cross flow filter element, optionally wherein the dynamic cross flow filter element is a rotational cross flow filter element comprising a ceramic membrane filter disc mounted on a shaft member.

92. The method of claim 91, wherein the rotational cross flow filter element comprises more than one filter disc and more than one shaft member.

93. The method of claim 79, wherein the sample of blood-derived plasma comprises fresh plasma, cryo-poor plasma or cryo-rich plasma, optionally wherein the sample of blood-derived plasma does not comprise a filter aid and/or has not been subjected to an alcohol or other fractionation processes.

94. The method of claim 79, further comprising adjusting the pH of the sample of blood-derived plasma prior to contact with the medium chain fatty acid.

95. The method of claim 79, wherein the medium chain fatty acid is selected from fatty acids of general formula CH3(CH2)nCOOH, wherein the fatty acid is a C-6 to C-12 carboxylic acid.

96. The method of claim 95, wherein the medium chain fatty acid is selected from enanthic (heptanoic) acid, caprylic (octanoic) acid, pelargonic (nonanoic) acid, capric (decanoic) acid, and salts and esters thereof.

96. The method of claim 79, wherein the fatty acid is caprylic (octanoic) acid and is mixed with the blood-derived plasma sample in an amount from about 0.30 g/g total protein in the sample to about 1.1 g/g total protein in the sample.

97. The method of claim 79, wherein step (a) comprises:
vigorously mixing the blood-derived plasma and medium chain fatty acid to obtain a homogeneous emulsion, wherein the mixing is conducted for a period of at least 10 minutes, and
incubating the mixture for a period of time of at least 20 minutes.

98. The method of claim 79, wherein the steps of the method are performed at a temperature of about 18° C. to about 37° C.

99. The method of claim 79, wherein the first filtrate comprising soluble immunoglobulins contains one or more of the following impurities: IgA, IgM, albumin, α (alpha)-2 macroglobulin, α (alpha)-1 anti-trypsin, a lipid, and a lipoprotein.

100. The method of claim 79, further comprising subjecting the first filtrate comprising soluble immunoglobulins to further processing comprising one or more selected from low pH treatment, anion exchange chromatography, immunoaffinity chromatography, virus filtration, virus inactivation, concentration, and formulation for administration.

101. A method for purifying albumin from plasma, the method comprising:
(a) in a first tank, mixing a sample of blood-derived plasma comprising albumin and immunoglobulins with a medium chain fatty acid under conditions comprising a pH of about 4.15 to about 4.25, to permit selective precipitation of albumin from the sample, thereby forming a suspension comprising soluble immunoglobulins and insoluble albumin;
(b) feeding the suspension into a first filtration unit comprising a dynamic filter element to obtain a first retentate comprising the insoluble albumin and a first filtrate comprising the soluble immunoglobulin; and
(c) recovering the first retentate comprising insoluble albumin.
